(19) United States
(12) Patent Application Publication
(10) Pub. No.: US 2010/0286069 A1
(43) Pub. Date: Nov. 11, 2010

(54) CATIONIC PEPTIDE FOR DELIVERING AN AGENT INTO A CELL

(76) Inventors: Yi-Yan Yang, Singapore (SG); Nikken Wiradharma, Sigapore (SG); Weiyang Seow, Singapore (SG)

Correspondence Address: KLARQUIST SPARKMAN, LLP, 121 SW SALMON STREET, SUITE 1600, PORTLAND, OR, 97204, US

(21) Appl. No.: 12/666,787

(22) PCT Filed: Jun. 27, 2008
(86) PCT No.: PCT/SG2008/000228

§ 371 Date: Dec. 24, 2009

Related U.S. Application Data
(60) Provisional application No. 60/929,471, filed on Jun. 28, 2007.
(60) Provisional application No. 60/960,968, filed on Oct. 23, 2007.

(51) Int. Cl. A61K 38/16; C12N 5/02; A61K 38/10; C07K 9/00; C07K 14/00; C07K 7/08

(52) U.S. Cl. 514/21.3; 435/375; 514/21.4; 530/322; 530/324; 530/326

(57) ABSTRACT

There is presently provided a triblock peptide comprising a hydrophobic amino acid block, a histidine block and a cationic amino acid block. The triblock peptide may be used to form a nanoparticle for delivery of an agent into a cell.

### CROSS-REFERENCE TO RELATED APPLICATIONS

This application claims benefit of, and priority from, U.S. provisional patent applications Nos. 60/929,471, filed on Jun. 28, 2007, and 60/960,968, filed on Oct. 23, 2007, the contents of which are both fully incorporated herein by reference.

### FIELD OF THE INVENTION

The present invention relates generally to cationic peptides and methods for delivering an agent, such as a nucleic acid molecule, into a cell.

### BACKGROUND OF THE INVENTION

Non-viral gene delivery into animal cells is an important and developing technology in the research fields of molecular biology and biomaterial research. Such technology has high potential application in the commercial area of large-scale recombinant antibody production, is a useful tool in the study of gene function and regulation in specific cell types, and is highly relevant to the improvement of therapeutic approaches for treating many hereditary human diseases.

In a typical non-viral gene delivery vector, a cationic-based material is generally used to condense foreign DNA into a DNA/vector complex. However, as the mechanism governing the gene delivery system consists of multiple steps (16), the success of non-viral gene delivery vector does not only depend on its ability to condense DNA but also depends on its buffering capability to absorb protons upon endocytosis of the complex, a mechanism which is widely known as “proton-sponge” effect for intracellular delivery of DNA (27).

However, one of the major drawbacks of the currently available delivery materials, including cationic polymers (17-19) and dendrimers (20), is the cytotoxic effects these materials have on target eukaryotic cells. Given the potential therapeutic and clinical uses of these materials, less cytotoxic materials are needed to carry genes into various cells without affecting inherent signaling pathways and systems.

In an attempt to address cytotoxic effect of the cationic polymer gene delivery systems, cationic oligopeptides have been studied for the same application (21-24). Cationic oligopeptides made from naturally occurring amino acids have recently been widely explored as non-viral vectors for gene delivery (1-4) due to their biodegradability, greater biocompatibility and ease of production as well as compositional control compared to polymer-based vectors. Existing designs for oligopeptides consist of either a combination of cationic arginine residues (for DNA binding) and a hydrophobic compound (e.g. stearyl or cholesterol to promote the cellular uptake of oligopeptide/DNA complexes (1)), or cationic lysine and pH-sensitive histidine moieties (for endolysosomal escaping) (2). However, the success of these materials is still limited possibly due to relatively lower cationic charge density in each molecule as compared to the cationic polymer counterparts (21).

Thus, there is a need for new non-viral delivery systems that are not cytotoxic and that provide for efficient delivery of nucleic acids and other therapeutic agents into cells.

### SUMMARY OF THE INVENTION

The present invention relates to peptides useful as a delivery system for delivering agents, including nucleic acid molecules, into cells. The peptides are triblock peptides, comprising a hydrophobic block, a histidine block and a cationic block.

The peptides of the present invention may bind nucleic acid molecules efficiently, and induce high gene transfection efficiency in various cell lines, which may be comparable to or better than levels obtained using polyethylenimine (PEI). Since the peptides of the present invention are formed from amino acids, they are biodegradable and less toxic than commercially available transfection reagents such as PEI and Lipofectamine.

The peptides, due to the amphiphilic nature provided by the combination of the hydrophobic block and the cationic (and thus hydrophilic) block, are capable of assembling into core/shell nanoparticles. The formation of the cationic core/shell nanoparticles increases positive charge density at the surface of the nanoparticle and promotes cellular uptake of substances such as nucleic acids.

Agents other than nucleic acid molecules, including therapeutic agents such as anticancer drugs, including doxorubicin, paclitaxel and peptides such as Int-H1-S6A,F8A (derived from H1 of c-Myc), may be chemically attached to the hydrophobic block of the peptide or physically encapsulated into the core of the peptide nanoparticles. Such loaded nanoparticles may be used alone, or may be used to co-delivery a nucleic acid molecule into a cell.

Thus, in one aspect, there is provided a triblock peptide comprising: a hydrophobic amino acid block, a histidine block and a cationic amino acid block.

The hydrophobic amino acid block may comprise alanine, methionine, valine, leucine, isoleucine, phenylalanine or tryptophan or any combination thereof. The cationic amino acid block may comprise arginine or lysine or any combination thereof. The blocks may be arranged in order of the hydrophobic amino acid block, the histidine block and the cationic amino acid block from the N-terminus to the C-terminus. The triblock peptide may comprise a sequence as set forth in any one of SEQ ID NOS: 1 to 5.

In another aspect, there is provided a nanoparticle comprising a triblock polymer of the invention.

In yet another aspect, there is provided a nanoparticle/agent complex comprising a nanoparticle of the invention and an agent to be delivered into a cell, the agent complexed with the nanoparticle.

The agent may be complexed with the nanoparticle via an electrostatic interaction, a hydrogen-bonding interaction or a hydrophobic interaction. The agent may be complexed to the exterior of the nanoparticle or may be complexed with the interior of the nanoparticle.

The nanoparticle/agent complex may comprise an additional agent and the additional agent may be complexed with the exterior or the interior of the nanoparticle.

In a further aspect, there is provided a method of delivering an agent into a cell comprising contacting a nanoparticle/agent complex of the invention with a cell so that the nanoparticle/agent complex is taken up into the cell.

The cell may be in vitro or in vivo. If the cell is in vivo, the method may further comprise administering the nanoparticle/agent complex to a subject, including a human.

In another aspect, there is provided a pharmaceutical composition comprising a triblock peptide of the invention or a nanoparticle/agent complex of the invention.

The pharmaceutical may further comprise a pharmaceutically acceptable carrier.

In another aspect, there is provided use of a nanoparticle/agent complex of the invention for delivering an agent into a cell of a subject or use of a nanoparticle/agent complex of the invention in the manufacture of a medicament for delivering an agent into a cell of a subject.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention:

FIG. 1: Rational peptide design and complex images. a. Schematic of the designed peptides with (1) octaarginine for DNA binding and membrane penetration, (2) histidine residues for acidic compartment escape and (3) a hydrophobic segment for enhanced cellular uptake. Colour scheme: grey=C; white=H; blue=N; red=O and green=sidegroups of either I, F or W. b. Light microscopic image of the bigger I/DNA complexes (N/P 15). c. Scanning electron microscopic image of the smaller complexes.

FIG. 2: DNA binding ability of the peptides and kinetics of DNA capture. Retardation of electrophoretic DNA migration by H₄R₈ (a) and I₅H₄R₈ (b) complexes at N/P ratios specified (0-30). c. Percentage of florescence remaining due to intercalation of PicoGreen into uncaptured/loosely-condensed DNA, after correcting for blanks and background. Inset is a magnification of the main graph. Error bars represent standard deviation. d. Protection of DNA from sonicative shearing and DNase digestion by I₅H₄R₈. Lane 1: naked DNA as control; 2: naked DNA sheared by sonication; 3: naked DNA digested with DNase; 5 and 7: complexes (N/P ratios of 15 and 30, respectively) after successive sonication and DNase treatment; 4 and 6: DNA protected and uncondensed from the peptide by trypsination after successive sonication and DNase treatment.

FIG. 3: DNA binding ability of peptides. Electrophoretic mobility of plasmid DNA in F₅H₄R₈ (F) or W₅H₄R₈ (W)/DNA complexes at N/P ratios specified (0-30).

FIG. 4: In vitro gene transfection. a-c. Luciferase expression level in HEK293, HepG2 and 4T1 cells, respectively, when transfected with various peptide/DNA formulations. d. Percentage of cells expressing GFP when transfected with selected peptides. PEI at different N/P ratios was used but only the best result achieved in each cell line is presented as a comparison (PEI peaked at N/P 20 for HepG2 and N/P 10 for HEK293 and 4T1). Error bars represent standard deviation.

FIG. 5: Luciferase expression level of HEK293 cells when transfected with peptide/DNA complexes formed at either sodium acetate (10 mM, pH 5.0) or PBS (10 mM, pH 7.0) buffer. Error bars represent standard deviation.

FIG. 6: Cell viability assays in HEK293, HepG2 and 4T1 cells for peptide/DNA complexes and PEI at various peptide/PEI concentrations specified. Pure peptide: F for HEK293 and HepG2; I for 4T1. Error bars represent standard deviation.

FIG. 7: In vitro and in vivo gene transfection. a. Effects of chloroquine and bafilomycin on luciferase expression level of HEK293 cells transfected with I. b. Luciferase expression levels in HEK293 and 4T1 cells (N/P ratio in parenthesis) transfected with the mixture, supernatant (Super) or sediment (Sedi) formulation of I/DNA complexes and the effect of chloroquine (chloro). c. GFP expression in 4T1 cells, d. Luciferase activity in 4T1 tumor-bearing mice. Error bars represent standard deviation.

FIG. 8: Contributions of supernatant and sediment of peptide F/DNA complexes to the toxicity of HEK293 cells. Error bars represent standard deviations.

FIG. 9: Plot of intensity ratio of I3 to I1 as a function of logarithm of AK27 concentration in PBS buffer (pH 7.4).

FIG. 10: Electrophoretic mobility of DNA in nanoparticle/DNA complexes. (a) AK27/pDNA and (b) AK32/pDNA. Lane 1, 2, 3, 4, 5, 6, 7, and 8 are DNA ladder, naked pDNA, complexes at N/P of 1, 2, 3, 4, 5 and 10, respectively.

FIG. 11: Enzymatic degradation of pDNA. (a) pure pDNA; (b) pDNA extracted from AK27/pDNA complexes; (c) pDNA extracted from AK32/pDNA complexes. (a) Lane 1, 2 and 3: DNA ladder, naked pDNA and naked pDNA treated with DNAse I for 10 min; (b) and (c) Lane 1, 2-5, 6-9 and 10-13 are naked pDNA, peptide/pDNA at N/P of 1, 20 and 40 with exposure time of 0, 10, 30 and 60 min respectively.

FIG. 12: Particle size (a) and zeta potential (b) of peptide/pDNA complexes.

FIG. 13: Cell viability after incubation with AK27 (a), AK32 (b), AK27/pDNA (c) and AK32/pDNA (d) complexes at different concentrations in comparison with PEI at N/P ratio of 10. Error bar signifies standard deviation of 8 replicates.

FIG. 14: Effect of complexation pH on luciferase expression of AK27 peptide in HEK293 cell line. Error bar signifies standard deviation of 6 replicates.

FIG. 15: Luciferase expression level of AK27 (a) and AK32/pDNA (b) complexes in HEK293, HepG2 and 4T1 cell lines. Error bar signifies standard deviation obtained from 6 replicates.

### DETAILED DESCRIPTION

Presently provided are novel amphilic triblock peptides. The peptides may assemble into core/shell nanoparticles, which nanoparticles may be used to deliver nucleic acid molecules and/or other agents such as therapeutic agents or diagnostic agents into a cell.

The peptide delivery materials were designed with three specific blocks to facilitate binding or complexation of an agent to be delivered to a cell, cellular uptake of the complex, and endosomal escape once taken into a cell. Thus, the novel peptides comprise a cationic block, a hydrophobic block and a histidine block.

The cationic block provides positive charges that allow for efficient binding of nucleic acid molecules or other agents that may be negatively charged or have a negatively charged surface or region. The cationic block also provides a hydrophilic region that may readily form an exterior shell of a core/shell nanoparticle.

The hydrophobic block is included in the presently described novel peptides to allow for cell membrane penetration by the peptide and any agent, including a nucleic acid molecule, which may be complexed with the peptide. If a nucleic acid molecule that contains a transgene is being delivered to a cell using the present peptides, the eventual level of transgene expression may be influenced by the degree of hydrophobicity of the hydrophobic block. The hydrophobic block provides a self-assembling core portion that promotes formation of the core/shell nanoparticles and which may serve to encapsulate agents that are hydrophobic or that have a hydrophobic portion.

Due to the pH sensitive nature of histidine at physiologically relevant pH, the histidine block provides a proton sponge effect, allowing for endosomal escape by the peptide and any complexed agent, once taken up into the cell and endocytosed.

Without being limited by theory, the formation of the nanoparticles from the novel triblock peptides before complexation with nucleic acid that is to be delivered to a cell may increase the cationic charge density at the surface of the nanoparticles, allowing for better complexation with nucleic acid and providing a stable structure to the nanoparticle/nucleic acid complexes for increased cellular uptake.

The novel triblock peptides and nanoparticles presently described are biocompatible due to their amino acid composition, and may be less toxic than other currently used delivery materials, such as PEI and Lipofectamine.

Thus, there is presently provided a peptide comprising a hydrophobic amino acid block, a histidine block and a cationic amino acid block.

The triblock peptide may include a modification on one or more amino acids. For example, the triblock peptide may be glycosylated, acylated, acetylated, carboxylated or hydroxylated or any combination thereof, on one or more amino acids. The C-terminal carboxy group may be amidated with an amino group.

The triblock peptide may comprise naturally occurring amino acids, including the twenty standard amino acids usually incorporated into proteins. The triblock peptide may comprise non-naturally occurring amino acids, including D-amino acids or synthetic amino acids. However, it will be appreciated that a triblock peptide that comprises naturally occurring amino acids may be more biocompatible, more biodegradable and less cytotoxic than a triblock peptide comprising non-naturally occurring amino acids.

In various embodiments, the peptide may consist essentially of a hydrophobic amino acid block, a histidine block and a cationic amino acid block, or may consist of a hydrophobic amino acid block, a histidine block and a cationic amino acid block.

As used herein, “consists essentially of” or “consisting essentially of” means that the triblock peptide includes one or more amino acids, including at one or both ends of one or more of the three blocks within the described peptide, but that the additional amino acids do not materially affect the function of the triblock peptide to deliver an agent to a cell. For example, the triblock peptide consisting essentially of a hydrophobic amino acid block, a histidine block and a cationic amino acid block may have one, two, three, four or five amino acids at one or both ends of the described hydrophobic amino acid block, the described histidine block and/or the described cationic amino acid block, provided that such a triblock peptide still possesses the ability to complex with an agent that is to be delivered into a cell and be taken up by a target cell.

The hydrophobic amino acid block comprises, consists essentially of or consists of hydrophobic amino acids, meaning amino acids having a hydrophobic side chain. A hydrophobic side chain is a side chain that is aliphatic in nature or that has an aliphatic portion, such that the side chain is not as readily solvated by water or aqueous solutions as compared to a hydrophilic amino acid side chain. The hydrophobic amino acid may be any amino acid having a hydrophobic side chain. In certain embodiments, the hydrophobic amino acid is selected from naturally occurring hydrophobic amino acids, including alanine, methionine, valine, leucine, isoleucine, phenylalanine or tryptophan.

The hydrophobic amino acid block may be composed of a single type of hydrophobic amino acid, for example all alanine residues, or may be a combination of two or more different types of hydrophobic amino acids, for example a combination of two or more of alanine, isoleucine, phenylalanine and tryptophan.

In certain embodiments, the hydrophobic amino acid block consists of alanine, isoleucine, phenylalanine or tryptophan, or any combination thereof.

The length of the hydrophobic amino acid block may vary, and may be 4 or more or from 4 to 20 hydrophobic amino acids in length. The length of the block may be chosen depending on the specific hydrophobic amino acids included in the block and the desired ability to assemble into a nanoparticle, to encapsulate an agent that is to be delivered into a cell and to effect the desired level of expression of a transgene once delivered into a cell. The hydrophobic amino acid block may be 4 or more, 5 or more, 10 or more, 12 or more, 15 or more, or 20 or more hydrophobic amino acids in length. Alternatively, the hydrophobic amino acid block may be from 4 to 20, from 5 to 20, from 10 to 20, from 12 to 20 or from 15 to 20 hydrophobic amino acids in length.

The histidine block comprises, consists essentially of or consists of histidine amino acids. Histidine has an imidazole side chain with a pKa of 6.0, and relatively small changes in cellular pH can effect the charge on the imidazole side chain. Thus, as stated above, histidine can act as a “proton sponge”, having a greater tendency to bind a proton when in the lower pH environment of the endosome and a greater tendency to release a proton upon entry into the cytosol, which may thus facilitate endosomal escape of the triblock peptide and any complexed agent.

The length of the histidine block may vary, and may be 3 or more or from 3 to 20 histidines in length. The histidine block may be 3 or more, 4 or more, 5 or more, 10 or more, 12 or more, 15 or more, or 20 or more histidines in length. Alternatively, the histidine block may be from 3 to 20, from 4 to 20, from 5 to 20, from 10 to 20, from 12 to 20 or from 15 to 20 histidines in length.

The cationic amino acid block comprises, consists essentially of or consists of cationic amino acids, meaning amino acids having a cationic side chain. A cationic side chain is a side chain that is positively charged at neutral pH or that has a pKa greater than 7. The cationic amino acid may be any amino acid having a cationic side chain. Preferably, the cationic amino acid is selected from naturally occurring amino acids, including arginine and lysine.

The cationic amino acid block may be composed of a single type of cationic amino acid, for example all arginine residues, or may be a combination of two or more different types of cationic amino acids, for example a combination of arginine and lysine.

In certain embodiments, the cationic amino acid block consists of arginine or lysine, or any combination thereof.

The length of the cationic amino acid block may vary, and may be 5 or more or from 5 to 25 hydrophobic amino acids in length. The length of the block may be chosen depending on the desired charged density on the surface of an assembled nanoparticle. The hydrophobic amino acid block may be 5 or more, 8 or more, 10 or more, 12 or more, 15 or more, 20 or more or 25 or more cationic amino acids in length. Alternatively, the cationic amino acid block may be from 5 to 25, from 8 to 25, from 10 to 25, from 12 to 25, from 15 to 25 or from 20 to 25 cationic amino acids in length.

Thus, the total length of the triblock peptide may be 11 amino acids or greater, 12 amino acids or greater, 15 amino acids or greater, 16 amino acids or greater, 17 amino acids or greater, 20 amino acids or greater, 25 amino acids or greater, 27 amino acids or greater, 30 amino acids or greater, 32 amino acids or greater, 35 amino acids or greater, 39 amino acids or greater, 40 amino acids or greater, 45 amino acids or greater, 50 amino acids or greater or 65 amino acids or greater. Alternatively, the total length of the triblock peptide may be from 11 to 65 amino acids, from 12 to 65 amino acids, from 15 to 65 amino acids, from 16 to 65 amino acids, from 17 to 65 amino acids, from 20 to 65 amino acids, from 25 to 65 amino acids, from 27 to 65 amino acids, from 30 to 65 amino acids, from 32 to 65 amino acids, from 35 to 65 amino acids, from 39 to 65 amino acids, from 40 to 65 amino acids, from 45 to 65 amino acids, from 50 to 65 amino acids, from 11 to 50 amino acids, from 15 to 45 amino acids or from 17 to 35 amino acids.

The triblock peptide, in various embodiments, may comprise: a hydrophobic amino acid block comprising from 4 to 20 hydrophobic amino acids, a histidine block comprising from 3 to 20 histidines and a cationic amino acid block comprising from 5 to 25 cationic amino acids; a hydrophobic amino acid block comprising from 4 to 20 hydrophobic amino acids, a histidine block comprising from 4 to 20 histidines and a cationic amino acid block comprising from 8 to 25 cationic amino acids; a hydrophobic amino acid block comprising from 5 to 20 hydrophobic amino acids, a histidine block comprising from 5 to 20 histidines and a cationic amino acid block comprising from 10 to 25 cationic amino acids; a hydrophobic amino acid block comprising from 10 to 20 hydrophobic amino acids, a histidine block comprising from 10 to 20 histidines and a cationic amino acid block comprising from 12 to 25 cationic amino acids; a hydrophobic amino acid block comprising from 12 to 20 hydrophobic amino acids, a histidine block comprising from 12 to 20 histidines and a cationic amino acid block comprising from 15 to 25 cationic amino acids; a hydrophobic amino acid block comprising from 15 to 20 hydrophobic amino acids, a histidine block comprising from 15 to 20 histidines and a cationic amino acid block comprising from 20 to 25 cationic amino acids; a hydrophobic amino acid block comprising from 4 to 10 hydrophobic amino acids, a histidine block comprising from 3 to 10 histidines and a cationic amino acid block comprising from 5 to 15 cationic amino acids; a hydrophobic amino acid block comprising from 10 to 15 hydrophobic amino acids, a histidine block comprising from 3 to 10 histidines and a cationic amino acid block comprising from 8 to 15 cationic amino acids; or a hydrophobic amino acid block comprising from 4 to 15 hydrophobic amino acids, a histidine block comprising from 3 to 10 histidines and a cationic amino acid block comprising from 5 to 15 cationic amino acids.

In particular embodiments, the triblock peptide comprises, consists essentially of, or consists of one of the following amino acid sequences:

| | |
|---|---|
| WWWWWHHHHRRRRRRRR; | [SEQ ID NO: 1] |
| IIIIIHHHHRRRRRRRR; | [SEQ ID NO: 2] |
| FFFFFHHHHRRRRRRRR; | [SEQ ID NO: 3] |
| AAAAAAAAAAAAHHHHKKKKKKKKKK; | [SEQ ID NO: 4] |
| and | |
| AAAAAAAAAAAAHHHKKKKKKKKKKKKKKK. | [SEQ ID NO: 5]. |

The blocks may be arranged within the triblock peptide in any order. In an embodiment, the triblock peptide is in the order of hydrophobic amino acid block, histidine block, cationic block, in the order from N-terminus to C-terminus.

The presently described novel triblock peptides may be synthesised using known peptide synthesis techniques, including chemical synthesis methods such as liquid- or solid-phase synthesis methods, including Fmoc and Boc synthesis methods.

The triblock peptide may thus be assembled into a nanoparticle. The nanoparticle may be formed using standard micellar assembly methods known in the art. For example, nanoparticles may generally be formed by dissolution techniques, dialysis techniques or by single emulsion techniques as are known in the art.

The triblock peptide may be designed so as to self-assemble into nanoparticles. Thus, inclusion of the triblock peptide in a suitable buffer solution at a concentration above its critical micellar concentration (CMC) may result in formation of nanoparticles. A skilled person can readily determine the CMC for the triblock peptide using standard techniques available in the art and as described in Example 2 set out below.

The assembled nanoparticles may form a core/shell structure such that the cationic amino acid block tends to be oriented toward the outer shell of the nanoparticle and the hydrophobic amino acid block tends to be oriented toward in the inner core of the nanoparticle.

The triblock peptides and nanoparticles may be used to delivery an agent into a cell, including a nucleic acid molecule.

Thus, there is presently provided a method for delivering an agent into a cell, comprising complexing a triblock peptide with the agent and contacting the triblock peptide/agent complex with the cell so that the triblock peptide/agent complex is taken up into the cell. The triblock peptide may be assembled into a nanoparticle and thus the method may comprise complexing a nanoparticle comprising a triblock peptide with the agent and contacting the nanoparticle/agent complex with the cell so that the nanoparticle/agent complex is taken up into the cell. Although the following description refers to methods using the nanoparticle formed from the triblock peptide unless otherwise specified, such descriptions are applicable to methods using the triblock peptide.

The nanoparticle is complexed with an agent. References herein to a “nanoparticle/agent complex” or references herein that the agent and nanoparticle “are complexed” or form a “complex” refer to an interaction between the nanoparticle and agent that is sufficiently stable to allow for the agent to associate with the nanoparticle in order to be delivered into a cell. References herein to “taken up into” a cell, “delivery into” a cell, or “delivered into” a cell, mean that the agent enters into the interior of the cell from the exterior of the cell, becoming localised in the cytosol or within an organelle of the cell.

The agent may be any agent that is desired to be delivered into a cell. For example, the agent may be any agent having a therapeutic or preventative effect or which effects a desired therapeutic result, that is to be delivered into a cell. For example, the agent may comprise a nucleic acid molecule, a protein, a small molecule, a drug, an antibiotic, a hormone, a cellular factor or a polypeptide.

The triblock peptides and nanoparticles are particularly suited to complex with and deliver nucleic acid molecules into a cell, and thus in certain embodiments the agent is a nucleic acid molecule.

The nucleic acid molecule may be any nucleic acid molecule, including a single stranded or double stranded nucleic acid molecule, and including DNA, RNA or DNA/RNA hybrids. For example, the nucleic acid molecule may be an siRNA, an miRNA, or a nucleic acid molecule encoding a transgene that is to be expressed within the cell. It will be appreciated that if the nucleic acid encodes a transgene that is to be expressed within the cell, the nucleic acid molecule should also include any necessary regulatory elements required to effect expression of the transgene under desired conditions within the particular cell type. The transgene may be a therapeutic transgene or may be a transgene involved in a diagnostic method.

Alternatively, the agent to be delivered to a cell may be a pharmaceutically active agent, a small molecule, a drug or a protein, including a polypeptide, an antibody, a full length protein, a protein fragment, a protein domain, a fusion protein, an oligopeptide or a peptide. In particular embodiments, the agent may be an anticancer agent including doxorubicin, paclitaxel and peptides such as Int-H1-S6A,F8A, derived from H1 of c-myc (sequence:

| | |
|---|---|
| RQIKIWFQNRRMKWKKNELKRAFAALRDQI. | [SEQ ID NO: 6]). |

If the agent to be delivered is a protein, the protein may be biologically active in that it possesses a biological function in certain biological contexts, such as enzyme activity, binding to a target molecule such as another protein or protein domain or a nucleic acid sequence, hormone activity, cell signalling activity, transcription activation or suppression activity, cell growth or cell cycle regulation, anti-cancer activity or cytotoxic activity.

It may be desirable to monitor the agent once delivered into the cell, and thus the agent may be coupled to a detectable label, for example a small molecule fluorescent tag, which allows for detection of the agent once taken up into living cells, in a non-invasive manner.

Detectable label refers, to any tag or label that can be detected by any means, directly or indirectly, for example by using visualizing methods, autoradiography methods, colour development methods or by affinity binding. It will be appreciated that the detectable label selected should not interfere with the ability of the agent to complex with the nanonparticle or to be taken up by a cell. For example, the tag or label may comprise a fluorescent group, a chemiluminescent group, a radioactive group, a ligand (for example biotin), a photolabile fluorescent group, a paramagnetic group, or a heavy metal complex or moiety.

The agent to be delivered into the cell is capable of interacting with the nanoparticle to form a complex. Thus, the agent may have a region or portion of the agent available to interact with the nanoparticle to form a complex. For example, the agent may form a complex with the nanoparticle via a hydrophobic, electrostatic or hydrogen bonding interaction between various functional groups available on the agent and complementary functional groups available on the exterior shell of the nanoparticle.

The agent may interact with the exterior shell of the nanoparticle, which is positively charged. Thus, in order to complex the nanoparticle with the agent that is to be delivered into the cell, the agent may be negatively charged or may have a negatively charged, or anionic, region such as a portion of the agent or a tag attached to the agent. The negative charges on the agent allow the agent to form a complex with the cationic nanoparticle via electrostatic interaction. For example, if the agent is a nucleic acid molecule, the negatively charged phosphates on the nucleic acid molecule backbone will be able to interact with the positive charges on the surface of the nanoparticle to form a nanoparticle/nucleic acid complex. If the agent is a protein, the protein may contain a region on the surface of the protein that is negatively charged due to the spatial arrangement of negatively charged amino acids at the surface of the protein. The protein may be designed as a fusion protein having a stretch of amino acids containing negatively charged amino acids, for example at the C-terminus of a biologically active protein or protein domain. Alternatively, the protein may be modified with a negatively charged group or tag attached to the protein. It will be appreciated that any modification, including by fusion of additional amino acids or by attachment of a negatively charged tag, should be done so as not to interfere with any biological function of the protein. Similarly, if the agent is a pharmaceutically active small molecule, the small molecule may have negatively charged functional groups or may also be modified with a negatively charged group or tag attached to the small molecule.

Alternatively, in order to complex the exterior of the nanoparticle with the agent that is to be delivered into the cell, the agent may be polar or charged or may have a polar or charged region such as a portion of the agent or a tag attached to the agent. The polar or charged groups on the agent allow the agent to form a complex with the cationic nanoparticle via hydrogen bonding interactions. For example, if the agent is a protein, the protein may contain a region on the surface of the protein that is polar or charged due to the spatial arrangement of polar or charged amino acids at the surface of the protein. The protein may be designed as a fusion protein having a stretch of amino acids containing polar or charged amino acids, for example at the C-terminus of a biologically active protein or protein domain. Alternatively, the protein may be modified with a polar or charged group or tag attached to the protein that contains functional groups capable of acting as a hydrogen bond donor or acceptor group. It will be appreciated that any modification, including by fusion of additional amino acids or by attachment of a polar or charged tag, should be done so as not to interfere with the biological function of the protein. Similarly, if the agent is a pharmaceutically active small molecule, the small molecule may have polar or charged functional groups or may also be modified with a polar or charged group or tag attached to the small molecule.

The agent may interact with the interior of the nanoparticle. Thus, in order to complex with the hydrophobic core of the nanoparticle, the agent may be hydrophobic or may have a hydrophobic region such as a portion of the agent or a tag attached to the agent. For example, if the agent is a protein, the protein may contain a region on the surface of the protein that is hydrophobic due to the spatial arrangement of hydrophobic amino acids at the surface of the protein. The protein may be designed as a fusion protein having a stretch of amino acids containing hydrophobic amino acids, for example at the C-terminus of a biologically active protein or protein domain. Alternatively, the protein may be modified with a hydrophobic group or tag attached to the protein. It will be appreciated that any modification, including by fusion of additional amino acids or by attachment of a hydrophobic tag, should be done so as not to interfere with the biological function of the protein. Similarly, if the agent is a pharmaceutically active small molecule, the small molecule may have negatively charged functional groups or may also be modified with a negatively charged group or tag attached to the small molecule.

More than one type of agent may be delivered at a time in the present methods. For example, the nanoparticle may be used to deliver a nucleic acid molecule complexed to the cationic exterior of the nanoparticle along with a pharmaceutical agent, such as an anticancer agent, complexed with the hydrophobic interior of the nanoparticle. Alternatively, if the pharmaceutical agent possesses sufficient negative charge or polar hydrogen bond donor or acceptor groups, the pharmaceutical agent may be complexed with the exterior of the nanoparticle in the same manner as the nucleic acid. Using a combination of agents complexed with the nanoparticle may allow for specific targeting and/or synergistic therapeutic effect in the cell.

Thus, in the present methods, the nanoparticle is complexed with an agent that is to be delivered into a cell, and optionally an additional agent. The nanoparticle may be complexed by addition of the nanoparticle to a solution containing the agent and optional additional agent (if to be complexed with the exterior of the nanop article).

The ratio of the mass of agent to mass of the nanoparticle may be optimized to ensure adequate formation of the complex and to provide a complex having the desired size and zeta potential, using routine laboratory methods. In certain embodiments, a mass ratio of nanoparticle: agent of about 0.2 or greater, or about 1 or greater, or about 2.5 or greater, or about 5 or greater, or about 10 or greater, or about 20 or greater, or about 30 or greater, or about 40 or greater, or about 50 or greater, allows for efficient, stable complex formation. Zeta potential, a measure of surface charge, may be used as a parameter to gauge complex formation. For example, a positive zeta potential, for example of about 5 mV or greater, from about 5 mV to about 20 mV, or from about 20 mV to about 100 mV may be used to indicate formation of an appropriate complex between the agent and the nanoparticle.

If the agent to be delivered is a nucleic acid, the N/P ratio may be used to form complexes of appropriate size and zeta potential. The N/P ratio refers to the relative molar content of nitrogen atoms (N) in each peptide molecule to that of the phosphate groups (P) in each nucleic acid molecule. Suitable complexes may be formed at N/P ratios of about 0.1 to about 100, of about 0.5 to about 5, of about 1 to about 40, of about 1, of about 10, of about 20, of about 30 or of about 40. Typically, there is a general decreasing trend in the particle size and an increasing trend in the zeta potential of the nanoparticle/nucleic acid complex with increasing N/P ratio. Higher N/P ratios may protect the nucleic acid from degradation when contained within the nanoparticle/agent complex.

Similar to particle size, the value of the zeta potential of the complexes may also influence the efficiency of gene expression. Ideally, high net positive charge after complexation is preferred to enhance the cellular uptake of the complexes, owing to the interaction between the negatively-charged cell membrane and the positively-charged complex particles. Therefore, the higher the net positive charges of the complexes, the more likely the complexes to be taken up by the cells. However, high net positive charges contained in the complexes may at the same time make the cell membrane more susceptible to being disrupted, causing cellular toxicity in vitro. Higher positive charge density on the surface of the nanoparticles may provide zeta potential of the nanoparticle/agent complex once formed.

Once a nanoparticle complexed to an agent is provided (including any optional additional agent), the complex may be delivered to a cell to allow for uptake of the agent into the cell.

Delivery to a cell comprises contacting the nanoparticle/agent complex with the surface of a cell. Without being limited to any particular theory, the nanoparticle/agent may be endocytosed by the cell, resulting in uptake of the nanoparticle/agent complex into the cell. Once inside the cell, the complex may be initially located within an endosome. In the endosome, at least some of the histidine residues within the nanoparticle may become protonated, causing the breakdown of the endosomal membrane and allowing the complex to escape from the endosome, thus releasing the agent into the cytosol.

The cell to which the agent is to be delivered may be any cell, including an in vitro cell, a cell in culture, or an in vivo cell within a subject. The term “cell” as used herein refers to and includes a single cell, a plurality of cells or a population of cells where context permits, unless otherwise specified. The cell may be an in vitro cell including a cell explanted from a subject or it may be an in vivo cell in a subject. Similarly, reference to “cells” also includes reference to a single cell where context permits, unless otherwise specified.

The cell may be derived from any organism, for example an insect, a microorganism including a bacterium, or an animal including a mammal including a human.

A skilled person can readily determine whether the agent has been delivered into the cell using known methods and techniques, including various detection methods, immunoassays and fluorescence labelling techniques. A skilled person can also readily determine whether the agent retains any biological, pharmaceutical or therapeutic function provided there exists a direct or indirect assay for that particular function within the cell.

There is also provided a nanoparticle/agent complex as described above, including a nanoparticle/agent complex containing an additional agent.

There is also provided use of the above-described nanoparticle/agent complex for delivering the agent into a cell, or use of the above-described nanoparticle/agent complex for the manufacture of a medicament for delivering the agent into a cell, including when the cell is an in vivo cell in a subject.

To aid in administration of such a nanoparticle/agent complex to a subject, the complex may be formulated as an ingredient in a pharmaceutical composition.

Therefore, there is provided a pharmaceutical composition comprising a nanoparticle/agent complex as described above. The pharmaceutical composition may further include a pharmaceutically acceptable diluent or carrier. The pharmaceutical composition may routinely contain pharmaceutically acceptable concentration of salts, buffering agents, preservatives and various compatible carriers. For all forms of delivery, the nanoparticle/agent complex may be formulated in a physiological salt solution.

The proportion and identity of the pharmaceutically acceptable diluent or carrier is determined by the chosen route of administration, compatibility with biologically active molecules such as nucleic acids or proteins if appropriate, and standard pharmaceutical practice.

The pharmaceutical composition can be prepared by known methods for the preparation of pharmaceutically acceptable compositions suitable for administration to subjects, such that an effective amount of the nanoparticle/agent complex and any additional active substance or substances is combined in a mixture with a pharmaceutically acceptable vehicle. An effective amount of nanoparticle/agent complex is administered to the subject. The term “effective amount” as used herein means an amount effective, at dosages and for periods of time necessary to achieve the desired result, for example to deliver the agent into the target cell or cell population within the subject.

Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the nanoparticle/agent complex, in association with one or more pharmaceutically acceptable vehicles or diluents, and contained in buffer solutions with a suitable pH and iso-osmotic with physiological fluids.

Under ordinary conditions of storage and use, such pharmaceutical compositions may contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences and in The United States Pharmacopeia: The National Formulary (USP 24 NF19) published in 1999. Alternatively, the complex may be formulated at a time sufficiently close to use by mixing the nanoparticle and agent solutions, without the need for preservatives.

When administered to a patient, the nanoparticle/agent complex is administered in an amount effective and at the dosages and for sufficient time period to achieve a desired result. For example, the nanoparticle/agent complex may be administered in quantities and dosages necessary to deliver an agent which may function to alleviate, improve, mitigate, ameliorate, stabilize, prevent the spread of, slow or delay the progression of or cure an infection, disease or disorder, or for example to inhibit, reduce or impair the activity of a disease-related enzyme. A disease-related enzyme is an enzyme involved in a metabolic or biochemical pathway, which when the pathway is interrupted, or when regulatory control of the enzyme or pathway is interrupted or inhibited, the activity of the enzyme is involved in the onset or progression of a disease or disorder.

The effective amount of nanoparticle/agent complex to be administered to a subject can vary depending on many factors such as the pharmacodynamic properties of the nanoparticle/agent complex, the mode of administration, the age, health and weight of the subject, the nature and extent of the disorder or disease state, the frequency of the treatment and the type of concurrent treatment, if any, and the concentration and form of the nanoparticle/agent complex.

One of skill in the art can determine the appropriate amount based on the above factors. The nanoparticle/agent complex may be administered initially in a suitable amount that may be adjusted as required, depending on the clinical response of the subject. The effective amount of nanoparticle/agent complex can be determined empirically and depends on the maximal amount of the nanoparticle/agent complex that can be administered safely. However, the amount of nanoparticle/agent complex administered is preferably the minimal amount that produces the desired result.

The pharmaceutical composition may be administered to a subject in a variety of forms depending on the selected route of administration, as will be understood by those skilled in the art. Non-oral routes are preferred, particularly if a bioactive agent is being administered simultaneously in the same form with the nanoparticle/agent complex. The composition of the invention may be administered surgically or by injection to the desired site. In different embodiments, the composition is administered by injection (parenterally, subcutaneously, intravenously, intramuscularly, by direct injection into a targeted tissue or organ etc.) directly at a desired site.

The present methods and uses are further exemplified by way of the following non-limited examples.

### EXAMPLES

### Example 1

Here, short triblock oligopeptides were designed (17 amino acid residues, i.e. F₅H₄R₈, I₅H₄R₈, W₅H₄R₈). These peptide vectors induced efficient gene expression in various cell lines at levels that are comparable or even superior to the gold standard of polyethylenimine (PEI). The functionality of each block is essential for achieving high transfection efficiency, and the eventual level of expression can be further influenced by the degree of hydrophobicity of the hydrophobic block. Notably, the peptide I₅H₄R₈ mediates gene expression in a mouse breast cancer model much more efficiently than PEI. This approach of incorporating both hydrophobic and pH-sensitive amino acids within a cationic peptide structure therefore results in useful vectors for gene delivery.

Materials and Methods

Materials: 3-(4,5-Dimethylthiazol-2-yl)-2,5-diphenyltetrazoliumbromide (MTT), branched polyethylenimine (PEI, M_{w}˜25,000), deoxyribonuclease I (DNAse), chloroquine and bafilomycin A1 were all purchased from Sigma (Singapore). PicoGreen dsDNA quantification kit was from Molecular Probes Inc. (Eugene, Oreg.).

Peptide Synthesis: The peptides were prepared by Fmoc-solid-phase synthesis at Anaspec (San Jose, U.S.A.) or GL Biochem (Shanghai, China) and were of >95% purity based on HPLC analysis. Fidelity of the products was reconfirmed using matrix-assisted laser desorption/ionization time of flight (MALDI-TOF) mass spectrometry (Autoflex II, Bruker Daltronics). The [M+H]⁺ found/expected for H, I, F and W was: 1814.9/1815.1; 2381.5/2381.9; 2551.5/2552.0; 2745.5/2747.2.

DNA binding and protection assays: Agarose gel electrophoresis was conducted to study the DNA binding ability of the peptides. The peptide/DNA complexes containing 2.5 μg of DNA per 50 μL of solution were formed at various N/P ratios. The complex solution (10 μL), together with 2 μL of loading dye (Promega), were then loaded into the wells of a 1.0 wt % agarose gel preloaded with 2 μL of ethidium bromide (per 50 mL of gel solution), and electrophoresed at 80 V for 50 minutes prior to imaging (ChemiGenius Q, Syngene).

To evaluate if the complexes were able to protect the DNA against physical and enzymic degradation, the complexes were first sonicated (VC750, Sonics and Materials Inc.) at 35% power for 15 seconds. The same samples were then subjected to a consecutive digestion step using DNase, according to the manufacturer's recommendations. After 15 minutes, DNase was inactivated by chelating with Ca²⁺ ions using EDTA. To separate DNA from the complexes, 10 μL of trypsin solution (10×) was added to 15 μL of the final sample and incubated overnight at 37° C. to completely digest the peptide molecules. Gel electrophoresis, as described above, was then carried out to image the bands of released DNA with respect to other control samples.

Kinetics of DNA capture: PicoGreen is a dye whose florescence is dramatically increased after intercalating into uncondensed DNA double strands. Upon condensation of the DNA, however, PicoGreen becomes excluded resulting in a drop in florescent intensity. Making use of this behavior, the kinetics of the DNA capturing process was traced and quantified. At time=0, the peptide solution was dripped into the DNA solution and vortexed for ˜10 seconds. At appropriate time intervals, 10 μL of the complex solution was taken out and mixed with 990 μL of PicoGreen working solution (diluted 400 times from its stock solution with TE buffer, pH 7.3). The fluorescence emission spectrum was then recorded with a Fluorolog LFI 3751 fluorescence spectrometer (Jobin Yvon Horiba). The excitation wavelength used was 480 nm and the silts were set to 4 nm. The fluorescence area intensity at any time point expressed as a percentage of the fluorescence intensity obtained with the initial concentration of DNA (before mixing with peptide) was used to reflect the concentration of uncaptured/loosely captured DNA. A profile was plotted to trace the temporal evolution of the DNA capturing process. Blanks and background were corrected for in all calculations. A calibration curve spanning the range of DNA concentration experienced throughout the experiments was achieved with an r²>0.99.

Scanning electron microscopy (SEM): SEM observations were carried out with a Jeol JSM-7400F microscope equipped with a field emission gun, at a magnification of 8,500× and an electron energy of 8 keV. The sample for imaging was prepared by depositing a drop of complex solution formed in de-ionised water onto the surface of a freshly prepared Si wafer. The wafer was then air-dried for 4 hours, before being mounted onto an Al sample holder and sputter-coated with Pt.

Cytotoxicity assay: Cells were seeded onto 96-well plates at a density of 6-10×10³ cells/well. After 24 hours, all media were replaced with fresh ones and 10 μL of sample was introduced into each well. The sample consisted of either pure peptide or peptide/PEI complexes dissolved in the medium at the desired concentration. After 4 hours, all media were aspirated and replaced with fresh ones. Cytotoxicity analysis was carried out 3 days later by first replacing all wells with fresh media and adding 20 μL of MTT (5 mg/mL in PBS). After incubating for another 3 hours, all wells were carefully aspirated and replaced with 0.15 mL of DMSO to dissolve the internalized purple formazan crystals. The solution was homogenised and the relative colour intensity was measured with a microplate reader (PowerWave X, Bio-Tek Inc.). A test wavelength of 550 nm and a reference wavelength of 690 nm were used. Cell viability was represented by the average of (absorbance_{550 nm}−absorbance_{690 nm}) of the sample expressed as a percentage of the intensity of the controls ±standard deviation. Each data point was repeated at least 8 times.

For complexes of F, I and W, a peptide concentration of 0.02 and 0.10 mg/mL will approximately correspond to N/P ratio 5 and 25, respectively. For H, a peptide concentration of 0.03 and 0.09 mg/mL will approximately correspond to N/P ratio 10 and 30. For PEI, a polymer concentration of 0.0029 and 0.0058 mg/mL will approximately correspond to N/P ratio 5 and 10. IC₅₀ values for the pure peptide and PEI complexes were read off at 50% cell viability.

In vitro luciferase and GFP expression: HEK 293 and HepG2 cells were maintained in Dulbecco's modified Eagle's medium (DMEM) and 4T1 cells were cultured in RPMI 1640 growth medium at 37° C. with 5% CO₂. All growth media used were completed with 10% fetal bovine serum, 100 μg/mL of streptomycin and 100 U/ml of penicillin.

One day before transfection, cells were seeded at a density that depended on the cell line and type of experiments. For the luciferase transfection assay, 0.5 mL of medium containing 5-8×10⁴ cells were seeded into each well of a 24-well plate, while 1.0 mL of medium containing 1.8-2.0×10⁵ cells were pipetted into each well of a 12-well plate for GFP expression. Peptide/DNA complexes were formed at various N/P ratios in 10 mM PBS (pH 7.0) by dripping equivolume solutions of peptide into DNA. The mixture was then vortexed for ˜10 seconds and left to stand for at least 30 minutes before usage. Meanwhile, all media were replaced with fresh ones. For experiments involving chloroquine or bafilomycin A1, growth medium containing a final concentration of 100 μM of chloroquine or 20 nM of bafilomycin A1 (dissolved in DMSO) was used. The complex solution (50 μL) containing 2.5 μg of the luciferase-encoded plasmids or 100 μL of complex solution containing 3.5 μg of GFP-encoded plasmids was subsequently dripped into each well. For experiments involving separation by size, the mixture was centrifuged at 3,000 rpm for 10 minutes before the supernatant and reconstituted sediment were separately dripped into the wells. All wells were completely aspirated and replaced with fresh media after 4 hours.

Analysis for luciferase expression was carried out after 3 days. Cells were washed once with 0.5 mL of PBS before the introduction of 0.2 mL of lysis buffer. Membrane lysis was completed with 2 freeze-thaw cycles and manual cell scratching between the cycles. The cell suspension was centrifuged at 14,000 rpm for 10 minutes. The supernatant (20 μl) was taken out, and mixed with 100 μL of a luciferase assay reagent (Promega) by vortexing. The relative light unit (RLU) was measured with a luminometer (Lumat LB 9507, EG&G Berthold) and normalized against the protein concentration as measured with a bicinchoninic acid protein assay kit (Pierce). Luciferase activity was finally expressed as RLU/mg protein ±standard deviation of at least 4 samples.

For the GFP transfection assay, cells were harvested after 2 days. Cells were first washed with 1.0 mL of PBS before 0.3 mL of trypsin was added to detach them. 0.3 mL of growth medium was then added, and the cell suspension was centrifuged at 14,000 rpm for 10 minutes. Two further cell-washing cycles of resuspension and centrifugation were carried out in PBS before fixation in 0.4 mL of 1% paraformaldehye. The percentage of cells expressing GFP was then determined using a flow cytometer (FACSCalibur, Becton Dickinson) from 10,000 events and reported as a mean±standard deviation of at least 4 samples.

pH effect on luciferase expression efficiency: HEK293 cells were transfected with peptide F/DNA complexes formed in either sodium acetate (10 mM, pH 5.0) or PBS (10 mM, pH 7.0) buffer. Each data point was from at least 4 repeats.

In vivo luciferase expression: Female Balb/c mice (Centre for Animals Resources, National University of Singapore) weighing 18-20 g were subcutaneously injected with 0.1 mL of serum-free RPMI 1640 containing 4×10⁵ 4T1 cells. A tumor was then allowed to develop in each mouse for 10-12 days. PEI and different formulations of I/DNA complexes were prepared as above, and 30 μl of sample containing 4 μg of DNA was injected directly into each tumor. The tumor tissues were harvested two days later. To analyse for luciferase activity, each tumor was first thoroughly homogenised in lysis buffer, followed by two further freeze-thaw cycles. The mixture was then centrifuged and assayed for its luciferase activity as described above. Expression level was given as RLU/mg protein ±standard deviation of at least 4 mice.

Results

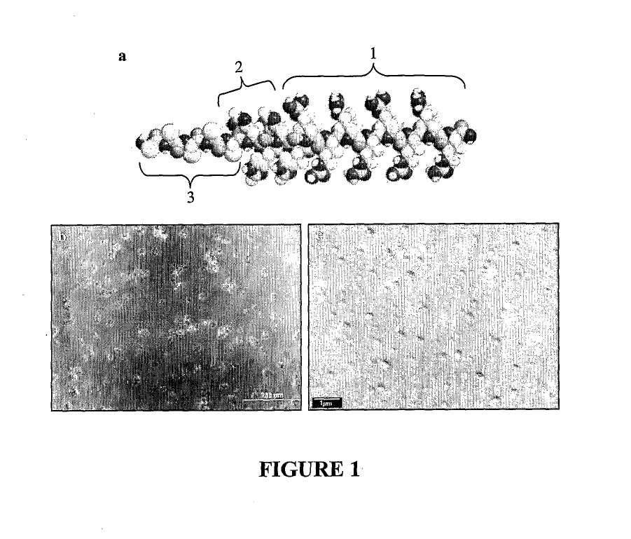

In this study, NH₂—I₅H₄R₈—CONH₂ (I), NH₂—F₅H₄R₈—CONH₂ (F), NH₂—W₅H₄R₈—CONH₂ (W) and NH₂—H₄R₈—CONH₂ (H) were prepared (FIG. 1a). The octaarginine block was factored into the peptide design for two main reasons. Firstly, with a pKₐ of ˜12.5, arginine becomes strongly protonated at physiological pH and is ideally suited for the ionic condensation of DNA. Secondly, it has previously been reported that oligoarginine exhibits cell penetrating and nucleus localization properties⁵, where the length of eight residues has been further optimized for the purpose of intracellular gene delivery (1). The histidine block was incorporated to aid the endosomal escape of peptides since like PEI, the immidazole sidegroups (plc ˜6.0) of histidine could provide proton scavenging effects upon internalization into acidic intracellular compartments of cells (6-8). A final design consideration concerns the addition of a hydrophobic block. Stearyl-modified octaarginine has been shown to mediate a near 100-fold increment in gene expression level in COS-7 cells when compared to octaarginine (1). This is likely to be due to an enhanced membrane penetration ability of the peptide/DNA complexes promoted by the hydrophobic stearyl segment. For better biocompatibility and degradability, however, the peptide was designed so that the hydrophobic block comprises pure amino acids such as isoleucine, phenylalanine or tryptophan (in order of decreasing hydrophobicity (9-11)).

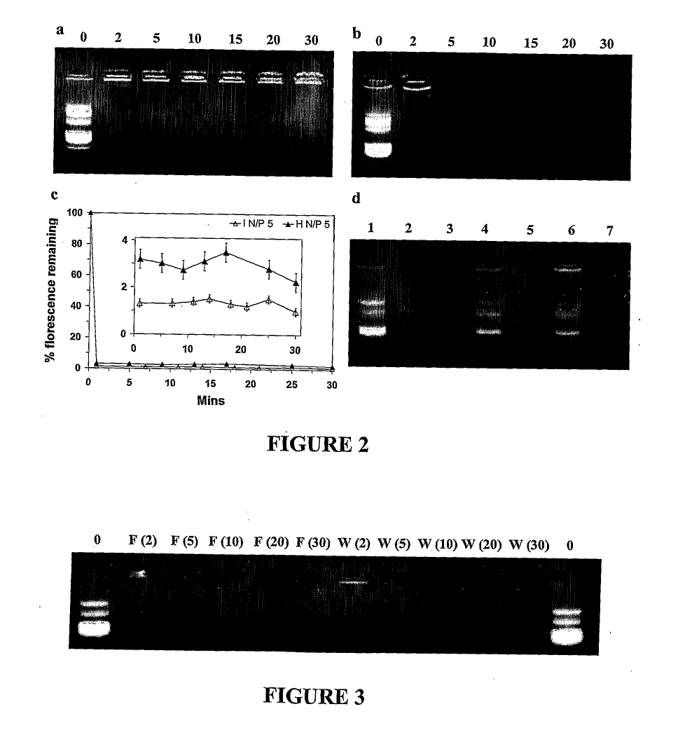

To evaluate the ability of the peptides to fully condense DNA so as to prevent its premature degradation en route to the nuclei of cells, plasmid DNA encoding the 6.4 kb firefly luciferase (pCMV-luciferase VR1255_C) driven by the cytomegalovirus (CMV) promoter (Carl Wheeler, Vical, U.S.A.) was bound to each peptide in 10 mM phosphate-buffered saline (PBS, pH 7.0) at various N/P ratios (defined here as the molar ratio of arginine residues in the peptide to the phosphorus content in DNA). Gel electrophoresis assays were then performed. It was observed that while bulk electrophoretic migration of DNA could be completely retarded by H at N/P 5 (FIG. 2a), some ethidium bromide molecules could still intercalate into the bound DNA, as evidenced by the brightly-fluorescing wells. In comparison, peptide I retarded bulk DNA movement at N/P 2 and also completely excluded ethidium bromide molecules from the condensed DNA from N/P 5 onwards (FIG. 2b). This indicates that I condensed DNA more strongly than H. Additionally, the zeta potential of pure H was determined to be lower than I (8.0±0.2 mV vs. 15.8±0.2 mV), which suggests that the hydrophobic groups might drive the aggregation of peptide molecules, thereby increasing the local concentration of positive charges for better DNA binding. Similar phenomena were also observed for peptides F and W (see FIG. 3).

To further understand the efficiency and kinetics of DNA capture by the peptides, PicoGreen was used as a sensitive fluorescence probe. The florescence of PicoGreen is dramatically increased upon intercalating into uncondensed DNA. This behavior can be employed to trace the temporal evolution of free/loosely bound DNA in solution. FIG. 2c reveals that the DNA-capture process was highly efficient, being completed in less than 1 minute for both H and I. Peptide I, however, quenched the fluorescence to a greater extent, which further proved that I was more capable of condensing DNA than H.

The ability of peptides to protect their DNA cargoes from physical and enzymic degradation was subsequently examined. Once formed, peptide/DNA complexes were sonicated and digested with DNase, before the condensed DNA was again released by trypsin digestion of its peptide carrier for imaging after electrophoretic migration (FIG. 2d). It is clear that unprotected DNA was extensively damaged by either sonication or DNase digestion. In sharp contrast, DNA released from the peptides was still intact, suggesting that condensed DNA benefited from being sterically shielded by the peptides from the effects of DNase and sonication.

The morphology and size distribution of peptide I/DNA complexes were next investigated. It was observed that under the current fabrication conditions, the I/DNA complexes showed a mainly bimodal distribution. Upon separation by centrifugation, the sediment was found to be constituted by large 15-30 μm particles (FIG. 1b) while the supernatant contained the smaller particles at 50-300 nm (FIG. 1c), as monitored by dynamic light scattering and light/scanning electron microscopy over a period of 5 hours. At high N/P ratios, it is possible that some hydrophobic blocks may radiate from the complex surfaces after the cationic block of the peptide has electrostatically interacted with DNA. This may, in turn, motivate the aggregation into large particles.

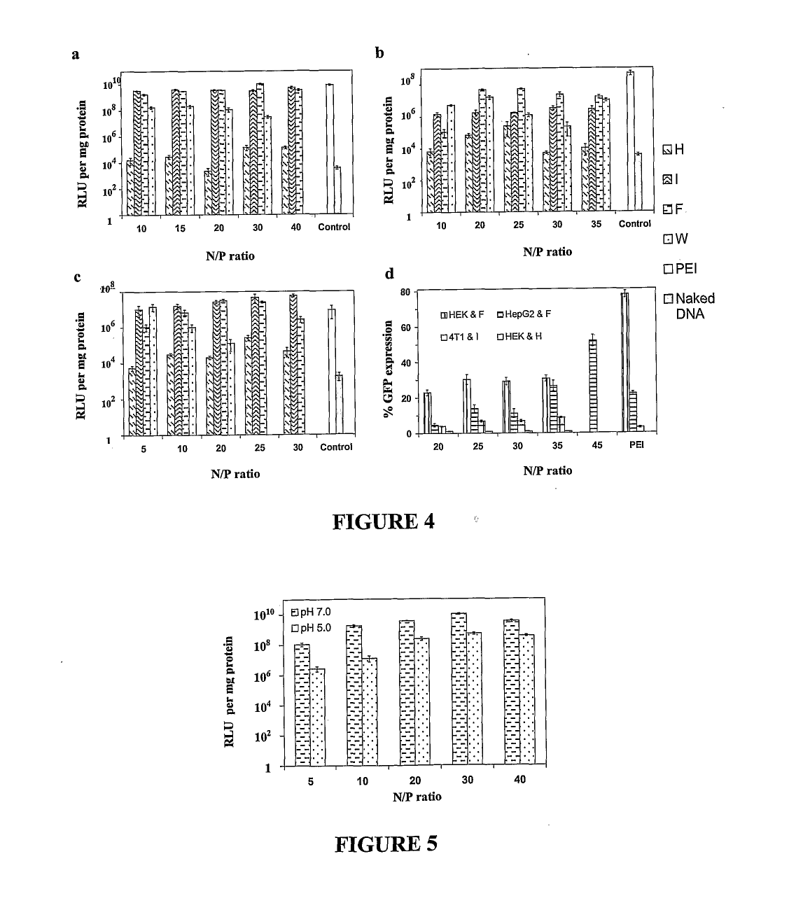

To examine the potential application of the peptides for gene delivery, in vitro transfection experiments were conducted on three cell lines—human embryonic kidney (HEK293) mammalian cell line, HepG2 human hepatocellular liver carcinoma cell line and 4T1 mouse breast cancer cell line (ATCC, U.S.A.) using either the luciferase reporter gene or the GFP reporter gene encoding the GFPmutl variant (pEGFP-C1) with 4.7 kb driven by the SV 40 early promoter (Clontech, U.S.A.). All cells were incubated with the peptide/DNA complexes for 4 hours. As expected, the final transfection efficiency depended strongly on the cell type, the peptide composition and N/P ratio of the complexes. FIG. 4a-c summarizes the luciferase expression levels. What was consistent in all three cell lines was that peptides I, F and W mediated luciferase expression levels that were 2-5 orders of magnitude higher than those obtained with peptide H (without a hydrophobic block). This may be due to the less efficient condensing of DNA by H, as suggested in FIG. 2a-c. Another possible explanation may be due to an enhanced degree of membrane association and penetration promoted by the hydrophobic blocks presented on the complex surfaces.

pH effect on lucerifase expression efficiency was studied using a pH 5.0 or pH 7.0 buffer. From FIG. 5, it can be seen that significantly higher expression levels were obtained with the complexes formed in PBS buffer. One possible reason was due to the protonation of histidine at pH 5.0, which then removed their proton-sponging functions.

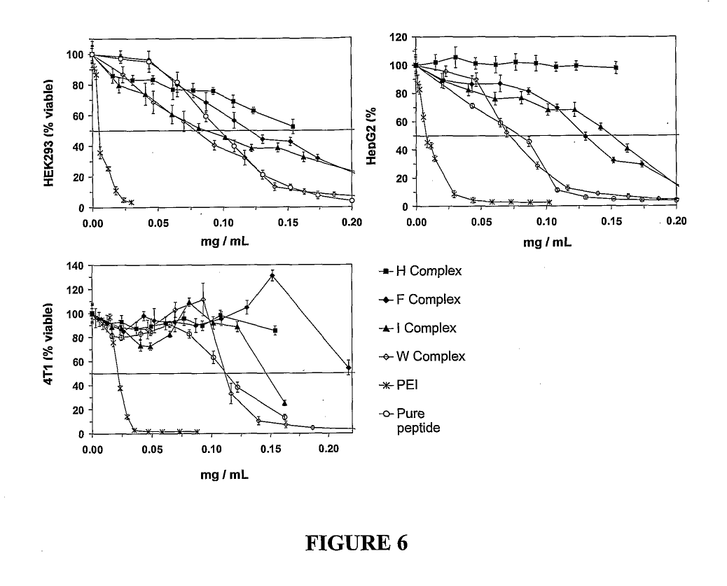

Significant differences in luciferase expression level were also observed by varying the degree of hydrophobicity of the hydrophobic block. Peptide W, being the least hydrophobic, constantly induced the lowest gene expression level in all cell lines tested. Peptide F, on the other hand, yielded the highest luciferase expression level in both HEK293 and HepG2 cell lines, while peptide I was optimal in 4T1 cell line. More encouragingly, the gene expression level achieved by the best peptide in each cell line was either superior or comparable to that obtained using PEI (branched, M_{w}=25,000), while causing less cytotoxicity (see FIG. 6). For instance, peptide I mediated a peak luciferase expression level in 4T1 cells that was 6.4 times higher than PEI with nearly 90% of the cells viable. Intrinsically, pure peptide I was also less toxic than PEI with an IC₅₀ value (defined as the concentration of pure I at 50% cell viability) that was 5.0 times lower. Similarly with HEK293 and HepG2 cell lines, F achieved an expression level that was 0.8 and 0.2 times that of PEI, but with a pure peptide IC₅₀ value that was 6.1 and 4.4 times lower, respectively.

FIG. 4d shows the proportion of cells transfected with the GFP gene using the best peptide (based on luciferase expression level) for each cell line. PEI transfected more HEK293 cells with the GFP gene, compared to the peptide carrier. However, the reverse was true for HepG2 and 4T1 cells. In the case of HepG2 cells, the higher GFP transfection efficiency observed with the peptide suggests that the uptake of the peptide/DNA complexes is higher than that of PEI/DNA complexes. Nonetheless, the intracellular delivery and expression of DNA mediated by the peptide may still be less effective compared to PEI, which then explains its lower overall gene expression level (i.e. the luciferase expression level). On a similar note, FIG. 4d also reveals that peptide F transfected more HEK293 cells with the GFP gene than H. This corroborates the earlier proposal that interactions between the hydrophobic amino acids and cell membrane could promote cellular uptake.

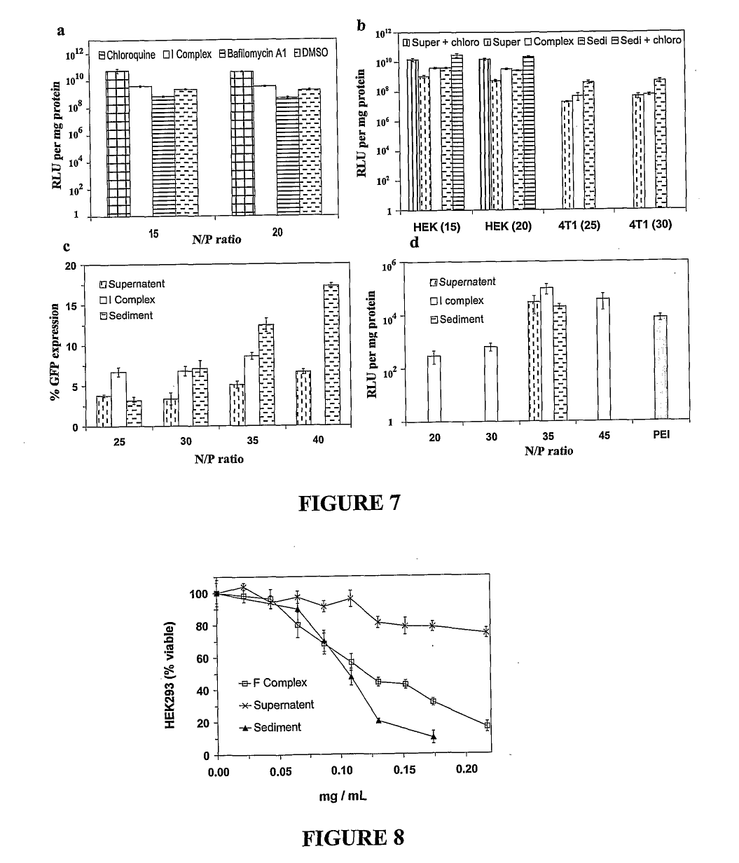

To evaluate the endosomal escaping property of the peptides, either chloroquine (a low-molecular weight drug known to buffer the pH of late endosomes and lysosomes) or bafilomycin (a specific inhibitor of the vacuolar type ATPase proton pumps found in many cells¹²) was added to the cell culture medium during transfection experiments. Typically, a superior transfection achieved in the presence of chloroquine is correlated with the lack of innate proton-sponging ability of the carrier itself. As revealed in FIG. 7a, such an increase in luciferase expression level was indeed detected when HEK293 cells were transfected with I/DNA complexes in the presence of chloroquine. However, a corresponding decrease was also observed when transfection was conducted in the presence of bafilomycin. Since transfection in the presence of pure DMSO (the solvent used to solubilise bafilomycin) did not reduce the expression level as significantly, it can be concluded that the main cause of reduction in transfection efficiency was due to the inhibition of proton-sponging activity by bafilomycin. This implies that while the histidine residues played an important role in proton scavenging, its length could be further increased to induce greater proton sponging abilities and gene transfection efficiencies.

Contributions of the large and small peptide/DNA complexes to the overall gene expression efficiency were investigated by transfecting 4T1 and HEK293 cells with either the supernatant or sediment of the centrifuged I/DNA complex solution. Interestingly, compared to the mixture formulation, the supernatant alone was able to mediate comparable luciferase expression levels in 4T1 cells (FIG. 7b). In HEK293 cells, however, the expression level was slightly lower. The larger sediment particles, on the other hand, performed well in HEK293 cells (comparable to the mixture), while in 4T1 cells, they induced higher expression levels. A similar trend was also observed when 4T1 cells were transfected with the GFP reporter gene (FIG. 7c). It has been commonly reported that big particles were better at mediating gene expression than smaller ones, partially because of a sedimentation effect (13, 14). In the present case, an additional reason may also be because the sediment contains ˜4-5 times more DNA than the supernatant.

Another observation was that either the supernatant or sediment alone could mediate high levels of expression, despite both carrying a lower DNA content compared to the entire mixture formulation (see FIG. 8). This suggests that the initial amount of DNA being introduced was not the main limiting factor amongst the multi-barrier process of gene expression, and also supports the general notion that only a small amount of DNA may be needed for effective in vitro expression. A trickier observation, however, was that the sediment, despite containing less DNA, still induced higher expression levels in 4T1 cells than the mixture. A possible explanation may be due to the relative speed of cellular uptake of the bigger and smaller particles. It was reported that smaller particles (<400 nm) were internalized more rapidly than bigger particles (up to ˜26 μm) (15). Thus in the mixture, there should be competitive uptake and although the smaller particles might get internalized preferentially, they were less effective in mediating gene expression. Consequently, the presence of smaller particles within the mixture maylimit the gene expression level when compared to the sediment formulation alone.

To understand the vesicle-escaping mechanism of the small and large DNA complexes, chloroquine was added to the sediment and supernatant formulations during transfection experiments in HEK293 cells. Doing so, the expression levels achieved by both the supernatant and sediment increased (FIG. 7b), suggesting that both the smaller and bigger complexes needed to escape from acidic compartments of the cells. While an endocytosis mechanism is plausible for the smaller particles, a less size-dependant pathway is probably needed to account for the internalization of the larger particles. It may be that the large aggregated particles fragment into relatively smaller ones before cellular uptake. However, a further study is necessary before a detailed mechanism can be proposed.

Finally, the in vivo gene transfection capacity of the peptides was evaluated by performing luciferase expression experiments with peptide I on mice bearing subcutaneous 4T1 breast tumors. In contrast to observations in vitro, the supernatant and sediment formulations mediated lower luciferase expression levels when compared to the mixture formulation (FIG. 7d). This may be because the amount of DNA being delivered by the supernatant and sediment formulations was smaller. The large DNA complexes might also not be uniformly distributed in the tumor, limiting the cellular uptake of the large complexes. Nevertheless, at the N/P ratio of 35, the luciferase expression levels achieved by the mixture, supernatant and sediment formulations were still about 13, 4 and 3 times higher than that obtained with PEI, respectively.

### Example 2

In this study, peptide amphiphile molecular constructs, (A)₁₂(H)₅(K)₁₀ (AK27) and (A)₁₂(H)₅(K)₁₅ (AK32), were designed from three blocks of amino acid residues, L-alanine, L-histidine, and L-lysine, and tested for efficacy as non-viral gene vectors. The amphiphilic constructs were able to self-assemble at concentration higher than 120 mg/L, which was estimated to be the CMC of the peptide in aqueous solution. The peptides were firstly characterized in terms of their particle sizes and zeta potentials. In the form of complex with plasmid DNA (pDNA), AK27/pDNA complex forms nanoparticles with the smallest size of around 248 nm at N/P ratio 40, whereas AK32/pDNA forms ones with the smallest size around 332 nm at N/P ratio 30. Similarly, the zeta potentials of the complexes were measured to be positive, and the highest surface charge of AK27/pDNA and AK32/pDNA were found to be 7.8 mV and 18.2 mV, both occurring at N/P ratio 40. In addition, peptide/pDNA complex nanoparticles formed from both AK27 and AK32 were also subjected to DNAse I enzymatic degradation, and it was observed that both peptides were able to protect pDNA from degradation, especially at higher N/P ratios. Furthermore, cytotoxicity of both AK27 and AK32 in the form of blank nanoparticles and pDNA complexes were tested and compared to that of high molecular weight PEI (at N/P 10). The two peptides showed minimum cytotoxicity, with cell viability of above 80%, even at higher N/P ratios, compared to PEI, which gave about 50% cell viability at N/P 10. Finally, to study the efficacy of the peptide amphiphilic nanoparticles as a gene carrier, transfection of luciferase encoding plasmid DNA (pLuc) were performed with HEK293 Human Embryonic Kidney, HepG2 Human Hepatocarcinoma, and 4T1 Mouse Breast cancer cell lines. In comparison with transfection induced by PEI at N/P 10, AK27 generally gave an order of magnitude less luciferase gene expression level than PEI, whereas AK32 generally induced almost comparable luciferase gene expression level. With such a comparable efficiency in inducing foreign gene expression to PEI, together with much less cytotoxic effects resulted compared to PEI, both AK27 and AK32 provide novel non-viral vectors for therapeutic gene delivery into animal cells.

Materials and Methods

Materials: Acetic acid, sodium acetate, sodium chloride, sodium dihydrogen phosphate, disodium hydrogen phosphate, polyethyleneimine (PEI, M_{w} 25 kDa), phenol, agarose, ethidium bromide, and 3-[4,5-dimethylthiazolyl-2]-2,5-diphenyl tetrazolium bromide (MTT) were all purchased from Sigma-Aldrich (France) and used as received. Phosphate buffered saline (PBS), Tris-boric acid-EDTA (TBE), Tris EDTA (TE) buffers, and 10% SDS were all purchased from 1^{st} BASE (Malaysia), and diluted to the intended concentration before use. Phosphate Buffer (PB) and sodium acetate buffer (SAB) used in this study were prepared at 10 mM and 20 mM, respectively. RQ 1 RNAse-free DNAse I enzyme, reporter lysis buffer, and luciferin substrate were purchased from Promega (U.S.A.), and pUC19 plasmid and DNA ladder were purchased from Fermentas (Canada). High glucose DMEM and RPMI 1640 growth media, fetal bovine serum (FBS), penicillin, streptomycin and Hoechst (fluorophore grade) were all purchased from Invitrogen Corporation (U.S.A.) and used for all the cell culture studies. Plasmid DNA encoding the 6.4 kb firefly luciferase (pCMV-luciferase VR1255C) driven by cytomegalovirus (CMV) promoter was provided by Car Wheeler, Vical (U.S.A.), which was amplified in E. coli DH5α and purified with Endofree Giga plasmid purification kit supplied by Qiagen (Dutch). HEK293, HepG2, and 4T1 cell lines were all obtained from ATCC (U.S.A.) and grown with the recommended conditions according to the supplier. Two amphiphilic peptides used in this study, (A)₁₂(H)₅(K)₁₀ (AK27) and (A)₁₂(H)₅(K)₁₅ (AK32), were designed by us and synthesized in GL Biochem (Shanghai) Ltd (P.R. China) at more than 95% purity.

Critical micelle concentration (CMC) measurement: The CMC of the peptide amphiphile was measured using pyrene as the probe fluorescence molecule. A series of peptide solutions was prepared at various concentrations (0.01 to 2000 mg/L) in PBS. When excited at 337 nm wavelength, the electrons in the pyrene molecules absorb energy, and in turn, emit photons at wavelengths between 350-450 nm, as indicated by five emission peaks in the pyrene fluorescence spectrum (30). At the concentration of amphiphile above its CMC, when the amphiphile forms core/shell structured nanoparticles having a hydrophobic core and a hydrophilic shell, the pyrene molecules aggregate and are solubilized into the core giving lower intensity of the third peak in the emission spectrum. Therefore, monitoring the intensity of the third peak of pyrene's emission spectra, usually normalized to the first peak's intensity, provides a means to estimate the transition concentration (i.e. CMC) of the amphiphile aggregation.

Complex formation of peptide amphiphiles with DNA: Complexes were formed at different N/P ratios by directly mixing equal volume of peptide and plasmid DNA (pDNA) solutions to achieve the intended N/P ratio, which measured the relative molar content of nitrogen atoms (N) in each peptide molecule to that of the phosphate groups (P) in each pDNA molecule, and was calculated based on the standardized 3 μgDNA/nmole DNA rule of thumb as described previously (31). To allow for complete electrostatic interaction between peptide and pDNA molecules, the solution was equilibrated at room temperature for 30 minutes upon mixing before being used for further studies.

Gel retardation assay: Various formulations of AK27/pDNA and AK32/pDNA complexes, ranging from N/P ratios of 1 to 10, were prepared in PBS. Post-equilibration, the complexes were electrophoresed on 1% agarose gel (stained with 4 μL of 0.5 μg/ml ethidium bromide per 50 mL of gel) in 0.5×TBE buffer at 80 V for 60 minutes. The gel was then analysed on a UV illuminator (Chemi Genius, Evolve, Singapore) to show the position of the complexed pDNA relative to that of the non-complexed form.

DNase degradation assay: In order to verify the ability of the peptide nanoparticles to protect pDNA from enzymatic degradation, complexes formed using AK27 and AK32 peptides at three different N/P ratios (N/P 1, 20, and 40) were incubated with equal volume (100 μL) of DNase I enzyme for different reaction times (0, 10, 30, 60) at 37° C. Post-incubation, DNA was then extracted with a mixture of phenol:chloroform solution (50:50 in volume, 200 μl). The extracted DNA was then precipitated with ice-cold absolute ethanol (700 μL), before being air-dried and re-dissolved in Tris-EDTA buffer (10 μL) for analysis. The DNA integrity was examined by 1% agarose gel electrophoresis assay as described above.

Particle size and Zeta potential measurement: The particle size and zeta potential of the complexes were measured by dynamic light scattering (Brookhaven Instrument Corp., Holtsville, N.Y., U.S.A.) and using Zetasizer (Malvern Instrument Ltd., Worchestershire, UK), respectively. Briefly, AK27/pDNA and AK32/pDNA complexes were formed in phosphate buffer (pH 6.5) at room temperature at different N/P ratios (i.e. 1, 10, 20, 30 and 40). All the particle size measurements were performed with a light source of 677 nm wavelength and 15° constant angle. The particle size measurement was repeated for 5 runs for each sample, and the data was reported as the effective mean diameter. The zeta potential measurement was repeated for 3 runs per samples, and the data was calculated automatically using the software from the electrophoretic mobility based on the Smoluchowski's formula.

Cell culture: Both HEK293 and HepG2 cell lines were cultured in high glucose DMEM medium supplemented with 10% fetal bovine serum (FBS), 100 μg/mL penicillin, and 100 μg/mL streptomycin at 37° C. in a humidified 5% carbon dioxide incubator. 4T1 cell line was also cultured with the same condition as the other cell lines, but in RPMI 1640 growth medium supplemented with 10 mM HEPES.

Cytotoxicity assay: The cytotoxicity of AK27, AK32, AK27/pDNA and AK32/pDNA complexes were all measured using the standard MTT assay protocol. Briefly, HEK293, HepG2 and 4T1 cell lines were seeded onto 96-well plates at 10,000 cells/well, 8,000 cells/well, and 6,000 cells/well seeding density, respectively. Post cell-seeding, the cells were incubated for one day before being used for cytotoxicity assay. For cytotoxicity assay with AK27/pDNA, complexes at N/P ratios of 1, 10, 20, 30, 35, 40, 45 and 50 were prepared in phosphate buffer (pH 6.5) as described earlier. For cytotoxicity assay with AK27 peptide, blank peptide solutions were prepared by mixing equal volume of peptide solution with buffer solution (instead of with DNA solution for complex formation). In terms of concentration units, AK27 blank peptide solutions translate to 39, 193, 387, 773, 967, 1160, 1353, 1547, 1740 and 1933 mg/L. Similarly, the concentrations of AK32 peptide were 27, 266, 532, 798, 931, 1064, 1197 and 1330 mg/L. The cells were then incubated with the peptide-containing solutions (20 μL of peptide in 200 μL of fresh growth media per well), or complex solutions (20 μL of complex in 200 μL of fresh growth media in a well) for 4 hours. After incubation of 4 hours, the cells were washed once with PBS buffer before being incubated for further 68 hours. On the third day, the cells were incubated for 4 hours with MTT solution in PBS (10 μL, 5 mg/mL) diluted with growth medium (1004). The medium was then removed and 150 μL of DMSO was added into each well to dissolve the formazan crystals. After homogenizing the solution, 100 μL of the samples was spectrophotometrically read in a microplate reader (Biorad) at 550 nm and 690 nm wavelength. The relative cell viability was measured as ([Abs₅₅₀]ₛₐₘₚₗₑ−[Abs₆₉₀]ₛₐₘₚₗₑ)/([Abs₅₅₀]_{control}−[Abs₆₉₀]_{control})×100%.

In vitro gene expression: HEK293, HepG2, and 4T1 cells were seeded onto 24-well plates at 80,000, 80,000 and 50,000 cells/well, respectively, using the same growth medium and conditions as described previously. After one day, the cells achieved about 70-80% confluence, at which transfection experiments were performed. Briefly, peptide/pDNA complexes were prepared at N/P 1, 10, 20, 30, 35, 40, 45 and 50, and different pH in either sodium acetate buffer (pH 5) or phosphate buffer (pH 6.5 and 7.4). The transfection pH was firstly optimized using HEK293 cell lines and AK27 peptide. The optimum pH was found to be around 6.5; and the subsequent transfection experiments were then performed with both AK27 and AK32 peptides for the three cell lines at pH 6.5. The total amount of DNA loaded was kept constant at 2.5 μg/well. Cells were allowed to incubate with serum and antibiotics-supplemented growth medium (500 μL/well), onto which the complex solutions (50 μL/well) were added. After four hours following the incubation with the complexes, the medium was then replaced with the fresh one. After 68 hours, the culture medium was removed, and the cells were washed with 0.5 mL of PBS (pH 7.4). 0.2 mL of reporter lysis buffer (Promega, Madison, Wis.) was then added to each well to lyse the cells. The cell suspension was next subjected to two cycles of freezing and thawing, and was centrifuged at 14,000 rpm for 5 minutes. 20 μL of the supernatant of the cell lysate was mixed with 100 μL of luciferase substrate (Promega). The relative light units (RLU) were measured using a luminometer (Lumat LB9507, Berthold, Germany). The luciferase activity was normalized to protein content using the BCA protein assay (Sigma). In all these experiments, both naked DNA-transfected cells and PEI-transfected cells were used as negative and positive controls, respectively. For the positive control, high molecular weight PEI (Mw 25 kDa) from Sigma-Aldrich was dissolved in water of HPLC grade to obtain a stock PEI solution at a concentration of 10 mg/mL, and used to form complexes at N/P 10 with pDNA according to the same complex formation protocol as the peptide complexes.

Results

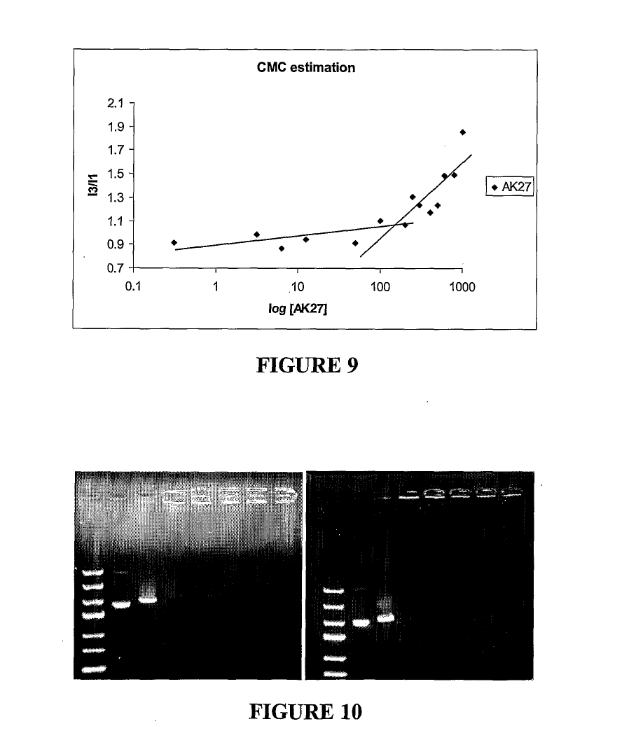

CMC Measurement: FIG. 9 shows the ratio of I3 to I1 against the logarithm of peptide concentration. The inflection point gives an estimate of CMC of AK27 in PBS (pH 7.4), which was approximately 120 mg/L.

DNA binding: Gel retardation assays of the two peptide-based amphiphiles were performed to qualitatively study the electrostatic interaction between the peptide and pDNA. As shown in FIG. 10, both peptides were able to provide enough positive charge density to electrostatically bind pDNA even at a low N/P ratio, at which peptide concentration was below its CMC. The complete retardation of DNA was achieved at N/P ratio of 3 for AK27 peptide and N/P ratio of 2 for AK32.

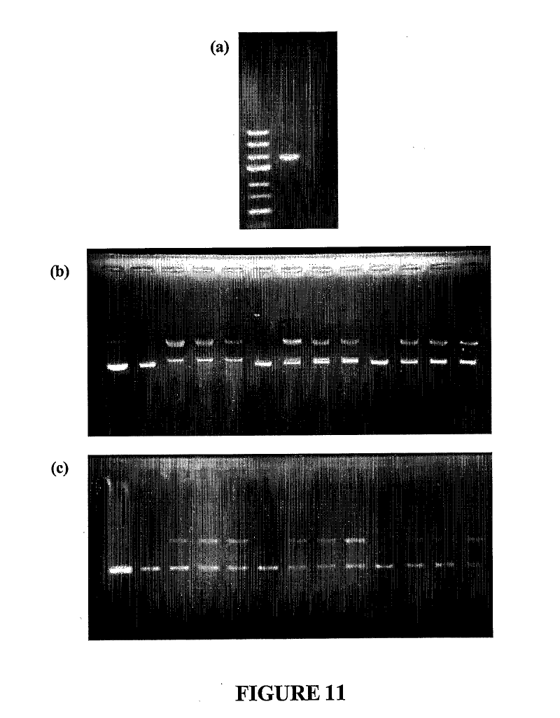

DNA degradation study: In the biological system, foreign DNA is subjected to enzymatic degradation by DNAses existed both in blood and in cell cytoplasm. It is therefore, necessary for the delivery vector to be able to condense such foreign DNA through electrostatic interactions and protect them from enzymatic degradation. For both peptides, degradation of pDNA in the peptide/pDNA complexes was studied by exposing the complexes (formed at N/P of 1, 20 or 40) with DNAse I enzyme. As a control, the naked pDNA was treated with DNAse enzyme for 10 minutes under the same conditions as the complex treatment. FIG. 11 provides gel images of the naked pDNA, extracted pDNA from AK27 complex, and extracted pDNA from AK32 peptide complex, after being treated with DNAse I enzyme for various timings.

Complete degradation of naked pDNA was observed after 10-minute incubation with DNAse I enzyme. After being complexed with AK27 or AK32 peptide, pDNA fragmentations were hardly observed even after exposure to DNAse I enzyme for 60 minutes when N/P ratio was higher than 20, indicating that complexation of pDNA with peptides at high N/P ratios efficiently prevented pDNA from degradation.

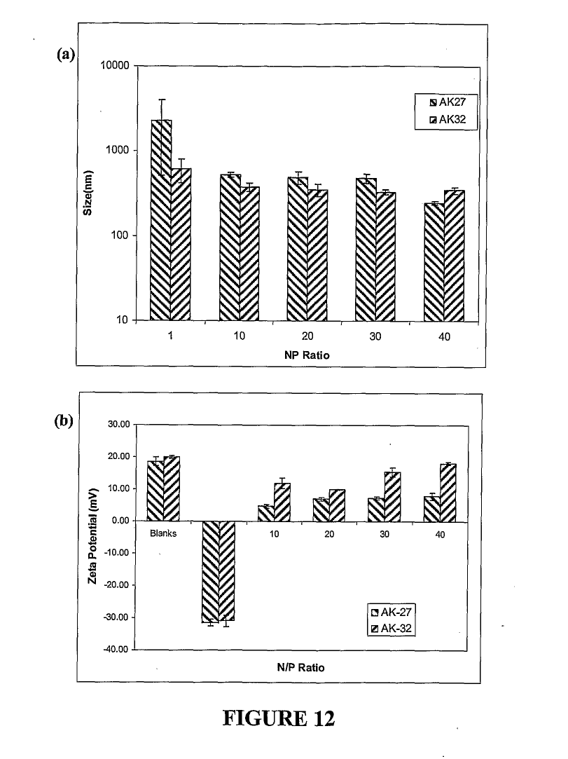

Particle size and zeta potential measurements: As shown in FIG. 12, there was a general decreasing trend in the particle size and an increasing trend in the zeta potential of the peptide/pDNA complexes as the N/P ratio was increased. pDNA was well condensed by the nanoparticles formed from both peptides. The smallest particle size measured by dynamic light scattering technique was around 248±11 nm for AK27/pDNA complexes at N/P ratio of 40, and 332±25 nm for AK32/pDNA complexes at N/P ratio of 30.

Similar to particle size, the value of the zeta potential of the complexes also affects the efficiency of gene expression. Ideally, high net positive charge after complexation is preferred to enhance the cellular uptake of the complexes, owing to the interaction between the negatively-charged cell membrane and the positively-charged complex particles. Therefore, the higher the net positive charges of the complexes, the more likely the complexes to be taken up by the cells. However, high net positive charges contained in the complexes may at the same time make the cell membrane more susceptible to being disrupted, causing cellular toxicity in vitro. For both peptides, the highest zeta potential achieved at N/P of 40 was measured to be approximately 7.8±1.1 mV and 18.2±0.5 mV. Compared to AK27, AK32 with a longer lysine block provided higher positive charge density on the surface of the peptide nanoparticles, leading to increased zeta potential of the peptide/pDNA complexes.

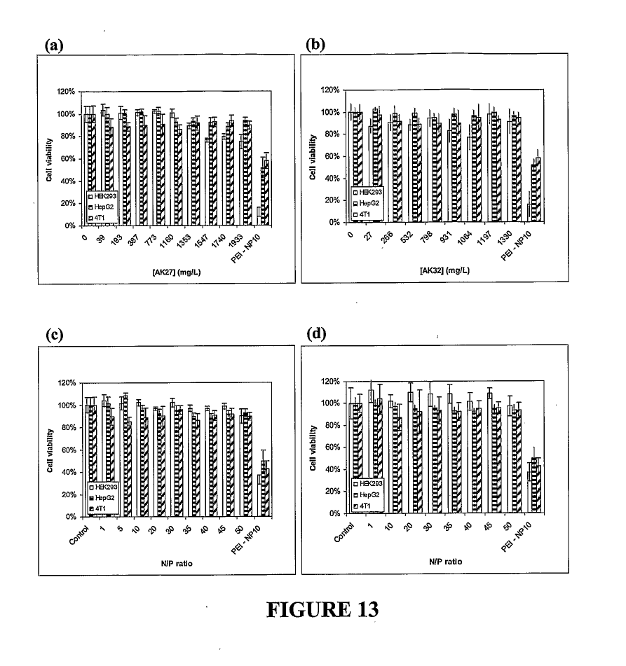

Cytotoxicity of peptide nanoparticles and their complexes with pDNA: Ideally, vector materials should not induce cytotoxic effect towards the cells. Furthermore, it should also be non-immunogenic to the body if it were to be used in vivo. FIG. 13 summarizes the cytotoxicity of peptide nanoparticles and their complexes with pDNA against HEK293, HepG2 and 4T1 cell lines at different concentrations. In general, both peptides had low toxicity to the cells, giving cell viability of about 80% or higher for almost all cell lines at concentrations of up to 1933 mg/L (FIGS. 13a and b). An increased length of lysine block did not increase the cytotoxicity of the peptide. Complexation with pDNA decreased the cytotoxicity of the peptides especially against HEK293 cell line tested at N/P ratio of up to 50 as shown in FIGS. 13c and d. This may be because the complexation of pDNA neutralized the positive charge of the peptide nanoparticles. Compared to PEI, the cytotoxicity of peptides and their pDNA complexes against the three cell lines was much lower.

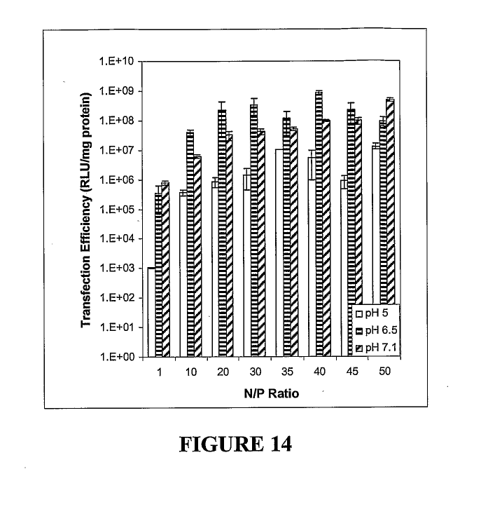

pH-dependent in vitro gene expression: Due to the difference in pKa values between histidine and lysine, there exists a pH-dependent gene expression of peptide nanoparticles. Since pKa of the imidazole side chain group of histidine lies around 6, at solution pH above this value, this side chain will be deprotonated. Similarly, since the pKa value of lysine's side chain is around 10, at solution pH below this pKa, lysine will be protonated, causing it to carry positive charges for DNA condensation. Therefore, there may be an optimum pH for gene expression. FIG. 14 summarizes the luciferase expression levels resulted from complexes formed at various pH values.

As can be seen from FIG. 14, the lowest expression efficiency was obtained when complexation was performed at pH 5. This was expected since at pH 5, both histidine and lysine would be protonated, leaving no available protonable groups to facilitate endosomal escape through ‘proton-sponge’ effect. When pH was changed to be above the pKa value of histidine, i.e., at pH 6.5 and 7.1, it was shown that expression efficiency became higher than that performed at pH 5, owing to the loss of buffering capacity of histidine at pH less than 6. More interestingly, luciferase transfection was found to be more efficient at pH 6.5 than at 7.1. The possible explanation for this is that at a lower pH, more protons were available in the solution to protonate the functional amine groups at the side chain of the lysine block. Therefore, even though histidine residues were present with buffering capacity to absorb proton at both pH 6.5 and 7.1, more of the lysine residues were protonated at the lower pH. This pH-dependent gene expression efficiency exhibits the equal importance of both buffering capacity and cationic property of the vector. From the fact that maximizing either buffering capacity (at the highest pH) or cationic property (at the lowest pH) alone did not make the transfection more efficient, it can be concluded that none of these mechanism are controlling the other, and that they co-exists with equal importance.

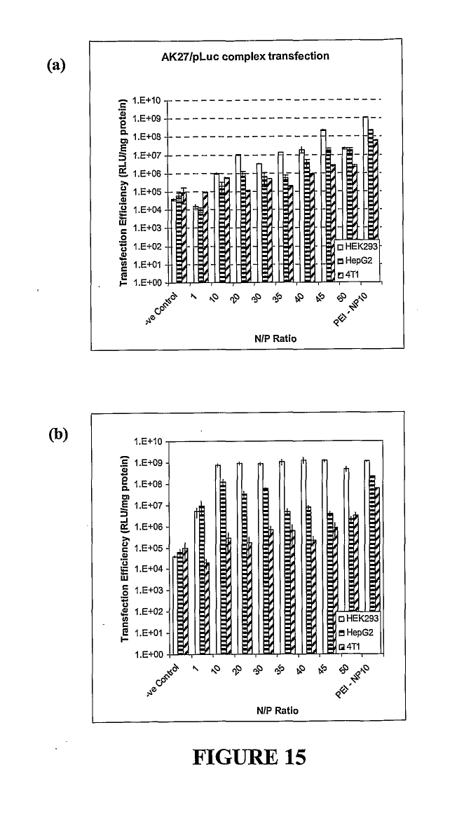

Luciferase expression in various cell lines: The luciferase expression efficiency induced by AK27 and AK32 peptides was tested in HEK293, HepG2 and 4T1 cell lines at different N/P ratios in comparison with PEI. N/P ratio of 10 was chosen for PEI because it provided less than 60% cell viability at this N/P ratio and increasing N/P ratio would significantly increase its cytotoxicity. In general, the luciferase expression efficiency induced by AK27 or AK32 in all the three cell lines increased with increasing N/P ratio as shown in FIG. 15. The highest luciferase expression level achieved by AK27 was at N/P ratio of 45, about one order of magnitude lower than that induced by PEI (FIG. 15a). However, the highest luciferase expression level achieved by AK32 with a longer length of lysine block appeared at a lower N/P ratio but was greater (FIG. 15b). This may be because the higher positive charge density of AK32/pDNA complexes induced greater cellular uptake. In addition, it was also observed from FIG. 15 that the highest luciferase expression level induced by AK32 was comparable to that provided by PEI in HEK293 and HepG2 cell lines but less than an order of magnitude lower in 4T1 cell line.

Thus, cationic nanoparticles were successfully self-assembled from amphiphilic peptides AK27 and AK32 in aqueous solutions. These nanoparticles possessed a strong DNA binding ability. The peptide/DNA complexes had an average size of 332 nm or less at N/P ratio of 30 to 40. Although the peptide nanoparticles and their pDNA complexes were positively charged, they had little cytotoxicity, which was lower than that of PEI. The peptide/pDNA complexes induced high luciferase expression level. Compared to AK27, AK32 with a longer length of lysine block formed pDNA complexes with greater positive charge density, providing higher luciferase expression efficiency, which was comparable to that yielded by PEI especially in HEK293 and HepG2 cell lines. Therefore, the cationic, biocompatible and biodegradable nanoparticles self-assembled from AK32 may provide a promising non-viral gene vector.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

1. Futaki, S. et al. Stearylated arginine-rich peptides: A new class of transfection systems. Bioconjugate Chem. 12, 1005-1011 (2001).
2. Yu, W. et al. Enhanced transfection efficiency of a systemically delivered tumor-targeting immunolipoplex by inclusion of a pH-sensitive histidylated oligolysine peptide. Nucleic Acids Res. 32, e48 (2004).
3. Trentin, D., Hall, H., Wechsler, S. & Hubbell, J. A. Peptide-matrix-mediated gene transfer of an oxygen-insensitive hypoxia-inducible factor-1α variant for local induction of angiogenesis. PNAS 103, 2506-2511 (2006).
4. Luo, D. & Saltzman, W. M. Synthetic DNA delivery systems. Nature Biotechnol. 18, 33-37 (2000).
5. Wender, P. A., Mitchell, D. J., Pattabiraman, K., Pelkey, E. T., Steinman, L. & Rothbard, J. B. The design, synthesis, and evaluation of molecules that enable or enhance cellular uptake: Peptoid molecular transporters. PNAS 97, 13003-13008 (2000).
6. Boussif, O. et al. A versatile vector for gene and oligonucleotide transfer into cells in culture and in vivo: Polyethylenimine. PNAS 92, 7297-7301 (1995).
7. Gabrielson, N. P. & Pack, D. W. Acetylation of Polyethylenimine Enhances Gene Delivery via Weaken Polymer/DNA Interactions. Biomacromolecules 7, 2427-2435 (2006).
8. Kim, Y. H., Park, J. H., Lee, M., Kim, Y. H., Park, T. G. & Kim S. W. Polyethylenimine with acid-labile linkages as a biodegradable gene carrier. J. Control. Release 103, 209-219 (2005).
9. Janin, J. Surface and Inside Volumes in Globular Proteins. Nature 277, 491-492 (1979).
10. Wolfenden, R., Andersson, L., Cullis, P. M. & Southgate, C. C. Affinities of Amino Acid Side Chains for Solvent Water. Biochemistry 20, 849-855 (1981).
11. Kyte, J. & Doolittle, R. F. A Simple Method for Displaying the Hydropathic Character of a Protein. J. Mol. Biol. 157, 105-132 (1982).
12. Akinc, A., Thomas, M., Klibanov, A. M. & Langer, R. Exploring polyethylenimine-mediated DNA transfection and the proton sponge hypothesis. J. Gene Med. 7, 657-663 (2005).
13. Rejman, J., Oberle, V., Zuhom, I. S. & Hoekstra, D. Size-dependent internalization of particles via the pathways of clathrin- and caveolae-mediated endocytosis. Biochem. J. 377, 159-169 (2004).
14. Green, J. J. et al. Biodegradable Polymeric Vectors for Gene Delivery to Human Endothelial Cells. Bioconjugate Chem. 17, 1162-1169 (2006).
15. Choi, H. S., Kim, H. H., Yang, J. M. & Shin, S. An insight into the gene delivery mechanism of the arginine peptide system: Role of the peptide/DNA complex size. Biochimica et Biophysica Acta 1760, 1604-1612 (2006).
16. Luo, D., Saltzaman, M. W., Nature Biotechnology 2000, 18: 33-36.
17. Christensen L. V., Chang C. W., Yockman J. W., Conners R., Jackson H., Zhong Z. Y., Jan F. J., Bu D. A., and Kim S. W., J. Controlled Rel., 2007, 118: 254-261.
18. Godbey W. T., Wu K. K., and Mikos A. G., PNAS 1999, 96: 5177-5181.
19. Kim J. H., Jeong J. H., Lee S. H., Kim S. W., and Park T. G., Journal of Controlled Release 2006, 116: 123-129.
20. Kono K., Akiyama H, Takahashi T., Takagishi T., and Harada A., Bioconjugate Chem. 2005, 16: 208-214.
21. Kim H. H., Lee W. S., Yang J. M., and Shin S., Biochimica et Biophysica Acta-Molecular Cell Research 2003, 1640: 129-136.
22. Futaki S, and Kawaguchi P. J. S. T., Biopolymers (Peptide Sci) 2006, 84: 241-249.
23. Bendifallah N., et. al., (2006) “Evaluation of cell-penetrating peptides (CPPs) as vehicles for intracellular delivery of antisense nucleic acid (PNA)”, Bioconjugate Chem. 17, 750-758.
24. Richard J. P., et. al. (2003) “Cell-penetrating Peptides, A reevaluation of the mechanism of cellular uptake”, J. Biol. Chem. 278, 585-590.
25. Lee E. S., Na K., and Bae, Y. H. (2005) “Super pH-sensitive multifunctional polymeric micelle” Nano letter 5, 325-329.
26. Midoux P., et. al., Som. Cell and Mol. Gen. 2003, 27: 27-47.
27. Gabrielson N. P. and Pack D. W., Biomacromolecules 2006, 7: 2427-2435.
28. Wang Y., Gao S. J., Wen H., Yoon H. S., and Yang Y. Y., Nature Materials 2006, 5: 791-796.
29. Wang Y., Wang L. S., Goh S. H. and Yang Y. Y., Biomacromolecules 2007, 8:1028-1037
30. Wolszczak M and Miller M. J Photochem Photobiol A Chem 2002, 147: 45-54.
31. Zeng, J. M., Too H. P., Ma Y. X., Luo E. S. E., and Wang S., Gene Med. 2004, 6: 1247-1256.

1. A triblock peptide comprising a sequence as set forth in any one of SEQ ID NOS: 1 to 5.

2. A nanoparticle comprising a triblock polymer of claim 1.

3. A nanoparticle comprising a triblock peptide comprising a hydrophobic amino acid block, a histidine block and a cationic amino acid block.

4. The nanoparticle of claim 3, wherein the hydrophobic amino acid block comprises alanine, methionine, valine, leucine, isoleucine, phenylalanine or tryptophan or any combination thereof, and the cationic amino acid block comprises arginine or lysine or any combination thereof.

5. The nanoparticle of claim 3, wherein the triblock peptide comprises, in order from the N-terminus to the C-terminus, the hydrophobic amino acid block, the histidine block and the cationic amino acid block.

6. A nanoparticle/agent complex comprising:
a nanoparticle of claim 3; and
an agent to be delivered into a cell, the agent complexed with the nanoparticle.

7. The nanoparticle/agent complex of claim 6, wherein the agent is complexed with the nanoparticle via an electrostatic interaction, a hydrogen-bonding interaction or a hydrophobic interaction.

8. The nanoparticle/agent complex of claim 6, wherein the agent is a nucleic acid molecule complexed to the exterior of the nanoparticle.

9. The nanoparticle/agent complex of claim 6, further comprising an additional agent.

10. The nanoparticle/agent complex of claim 8, wherein the additional agent is included in the interior of the nanoparticle.

11. The nanoparticle/agent complex according to claim 8, wherein the additional agent is complexed to the exterior of the nanoparticle.

12. A method of delivering an agent into a cell comprising:
contacting a nanoparticle/agent complex of claim 6 with a cell so that the nanoparticle/agent complex is taken up into the cell.

13. The method of claim 12, wherein the cell is in vitro.

14. The method of claim 13, wherein the cell is in vivo and the method further comprises administering the nanoparticle/agent complex to a subject.

15. The method of claim 14, wherein the subject is a human.

16. A pharmaceutical composition comprising a nanoparticle of claim 3.

17. The pharmaceutical composition of claim 16, further comprising a pharmaceutically acceptable carrier.

18. (canceled)

19. (canceled)

20. (canceled)

21. A nanoparticle/agent complex comprising:
a nanoparticle of claim 2; and
an agent to be delivered into a cell, the agent complexed with the nanoparticle.

22. A method of delivering an agent into a cell comprising:
contacting a nanoparticle/agent complex of claim 21 with a cell so that the nanoparticle/agent complex is taken up into the cell.

23. A pharmaceutical composition comprising a nanoparticle of claim 2.


SEQUENCE LISTING (6 sequences)

SEQ ID NO: 1 - protein, 17 aa, artificial
  synthetic peptide
WWWWWHHHHRRRRRRRR

SEQ ID NO: 2 - protein, 17 aa, artificial
  synthetic peptide
IIIIIHHHHRRRRRRRR

SEQ ID NO: 3 - protein, 17 aa, artificial
  synthetic peptide
FFFFFHHHHRRRRRRRR

SEQ ID NO: 4 - protein, 27 aa, artificial
  synthetic peptide
AAAAAAAAAAAAHHHHHKKKKKKKKKK

SEQ ID NO: 5 - protein, 32 aa, artificial
  synthetic peptide
AAAAAAAAAAAAHHHHHKKKKKKKKKKKKKKK

SEQ ID NO: 6 - protein, 30 aa, artificial
  synthetic peptide
RQIKIWFQNRRMKWKKNELKRAFAALRDQI